(19) United States
(12) Patent Application Publication
(10) Pub. No.: US 2026/0193662 A1
(43) Pub. Date: Jul. 9, 2026

(54) PROTEINS WITH MINIMAL N-TERMINAL INITIATOR METHIONINE

(71) Applicant: Unichem Laboratories Limited, Mumbai, Maharashtra (IN)

(72) Inventors: Dhananjay Sathe, Thane (IN); Saravanakumar Iyappan, Hyderabad (IN)

(21) Appl. No.: 19/130,824

(22) PCT Filed: Nov. 27, 2023
(86) PCT No.: PCT/IB2023/061911

§ 371 Date: May 16, 2025

(30) Foreign Application Priority Data
Nov. 29, 2022 (IN) 202221068864

(51) Int. Cl. C12N 15/70; A61K 38/48; C07K 14/375; C12N 9/48

(52) U.S. Cl. CPC C12N 15/70; A61K 38/4813; C07K 14/375; C12N 9/485; C12Y 304/11018

(57) ABSTRACT

The present invention relates to a recombinant plasmid, methods and compositions for the expression of recombinant proteins with minimal N-terminal initiator methionine. The present invention also provides strategies for efficient removal of N-terminal initiator methionine in recombinant proteins expressed at industrial scale and provides method for producing proteins with minimal N-terminal initiator methionine.

### FIELD OF THE INVENTION

The present disclosure generally relates to biotechnology field. Particularly, the present invention relates to a recombinant plasmid, methods and compositions for the expression and purification of recombinant proteins with minimal N-terminal initiator methionine.

### BACKGROUND OF THE INVENTION

A revolution in industrial biotechnology was sparked by the discoveries of the DNA structure and the development of recombinant DNA technology. Traditional microbiology techniques were merged with molecular biology to yield improved recombinant processes for the industrial production of primary and secondary metabolites, protein biopharmaceuticals and industrial enzymes. The recombinant DNA technology has demonstrated that desirable characteristics of the proteins synthesized industrially can be improved by controlling the expressions of target genes.

Due to the unique biosynthetic production process and molecular characteristics of biotechnological and biological products, the drug substance includes several molecular entities or variants; therefore, the desired product can be a mixture of anticipated post-translationally modified forms (e.g., glycoforms). Heterogeneity can also be produced during manufacture and/or storage of the drug substance or drug product. These forms may be active/inactive, and their presence may have deleterious effect on the safety and efficacy of the biological product.

Protein synthesis is initiated by formylmethionine (N-terminal methionine) in prokaryotes, and methionine in eukaryotes. During expression of recombinant proteins, N-terminal methionine is co-translationally cleaved by endogenous Methionine Aminopeptidase (MAP). The said cleavage process is not effective as quantity of recombinant protein expressed outperforms the capacity of limited amount of MAP to cleave the N-terminal methionine, thereby a substantial amount of expressed recombinant protein contains methionine as first amino acid (N-terminal initiator methionine), which is not part of the mature protein.

Further, recombinant proteins with N-terminal initiator methionine are prone to oxidation during production and storage, and this oxidation must be carefully watched and protected. Methionine sulfoxide or methionine sulfone can be produced when methionine residues in proteins are oxidised. Increased immunogenicity, inactivity, and aggregation may result from methionine oxidation. Methionine oxidation may limit the therapeutic product's clinical efficacy, stability, and regulatory acceptance.

The biological products having N-terminal methionine impurities may not have the same structure, activity, and stability as that of the native protein; and hence when administered in a human subject may result in unexpected immune response from the subject resulting in ineffective therapeutic functions. Therefore, it is important that the methionine impurities should be removed or minimized from the biological product before formulation.

For the preparation of recombinant protein with an innate N-terminus, various attempts have been made to remove the N-terminal Methionine. Predominantly, cyanogen bromide is used to cleave N-terminal methionine under extreme acidic conditions, but the method is limited to proteins without any internal Methionine residues. Also, the use of extreme acidic conditions may have deleterious effects on the protein of interest and hence, alternative method for removal of N-terminal Methionine needs to be explored.

Other strategies employed by researchers in past includes change of amino acid at second position (adjacent position to Methionine) for efficient cleavage of methionine, but the said strategy may lead to structural and functional changes in the molecular structure, thereby resulting in an altered molecule with undesirable effects.

Also, researchers have experimented with in-vitro digestion with purified methionine aminopeptidase (Miller et al., 1987) for removal of N-terminal Methionine. However, said methods lead to an additional step of purification and enzyme cost, hence economically not viable.

The N-terminal methionine can be co-translationally cleaved by the enzyme methionine aminopeptidase (MAP). In eukaryotes, methionine is removed either by cleavage of N-terminal signal peptide used for secretion, or by MAP. In prokaryotes, formylmethionine is first removed by formylmethionine deformylase resulting in N-terminal methionine which is then processed by MAP. In E coli, only one copy of MAP gene is present which is responsible for N-terminal methionine removal of 70% proteins expressed in E coli. Especially in E coli, as recombinant proteins are expressed on a large scale, the N-terminal methionine is retained in approximately 30% of the expressed protein, presumably due to saturation of MAP. (Refer: Paul Wingfield et al 2018 “N-Terminal Methionine Processing”); and hence researchers have focused their interest on co-expression of MAP via genetic manipulation of E coli. Various combination of promoters has been used for co-expression of MAP and protein of interest for removal of Methionine impurities.

Neupogen® (Filgrastim, r-met-huG-CSF) a recombinant protein expressed in E coli has been approved by USFDA for use in patients suffering from neutropenia during or after chemotherapy. Neupogen® is composed of a polypeptide chain of 175 amino acids in length (Souza et al., 1986; Lu et al., 1989b). Direct expression of the protein in E. coli leads to the accumulation of large quantities of expressed product in inclusion bodies. It has been reported that a typical Neupogen® production lot comprises of formylmethionine-G-CSF (f-metG-CSF) at very low (~1%) levels. Similarly, the oxidized forms represent less than 2% of total Neupogen®. [Ref: Alan C. Herman et al research titled “Characterization, Formulation, and Stability of Neupogen® (Filgrastim), a Recombinant Human Granulocyte Colony Stimulating Factor published in Formulation, Characterization, and Stability of Protein Drugs, Rodney Pearlman and Y. John Wang, eds., Plenum Press, New York, 1996].

The US patent U.S. Pat. No. 6,071,718A disclosed use of TAC promoters for expression of MAP and T7 promoters for expression Gene of interest. This patent also disclosed the use of methionine aminopeptidase cleaving N-terminal amino acids of the expressed protein (Beta casein). The patent further discloses requirement for expression of imino and amino peptidase and use of two proline amino acid for efficient cleavage of Methionine initiator. The addition of two prolines may add to undesired impurities or flanking amino acids at the N-terminus of in the expressed protein. Addition of amino acids to recombinant proteins may further alter the structure and function of the molecules expressed.

The US patent U.S. Pat. No. 11,060,123B2 disclosed use of TAC promoters for expression of MAP and T7 promoters for expression Gene of interest. In said patent, MAP gene is inserted into the genome of E coli, restricting the expression of protein of interest with use of said modified host only, more specifically E coli (Bl21 Gormet strain only). Further, only one copy of MAP is integrated in a single location of gene (gor) as a replacement, which also carries additional chloramphenicol gene for selection, restricting the availability of MAP for cleavage and removal of N-terminal Methionine in expressed proteins. As the genome of E coli is re-engineered, the said scheme limits the application to a particular strain of E coli, and hence, could not be used for protein expression in other strains of E coli.

The EP patent EP2430041B1 disclosed a modified lectin protein with 141 amino acids have been expressed in E coli as stable and soluble protein using recombinant DNA technology. The said modified lectin is similar to the native lectin with regards to the sugar binding specificity and the apoptotic properties enabling them to be used as drug delivery agents for cancer treatment and application as diagnostic tools.

Substantial amount of purified recombinant protein contains methionine as a first amino acid, which is not a part of mature protein sequence, i.e., an undesirable part for most of the therapeutic proteins. At present, there are no universally accepted strategies for efficient or complete removal of N-terminal methionine in recombinant proteins expressed at industrial scale. Therefore, protein expression strategies with high rate of expression/yield along with minimal impurities and simplified purification processes are solicitated.

### SUMMARY OF THE INVENTION

The present disclosure relates to a recombinant plasmid, methods and compositions for the expression and purification of recombinant proteins with minimal N-terminal initiator methionine. Further, the present invention provides strategies for efficient or complete removal of N-terminal initiator methionine in recombinant proteins expressed at industrial scale. The present invention further provides a recombinant plasmid comprising of a DNA construct for expression of Methionine Aminopeptidase (MAP) that enables efficient removal of N-terminal initiator methionine in expressed protein. The present invention also provides method for producing proteins with minimal N-terminal initiator methionine.

### In an Aspect, the Present Invention Relates to a Recombinant Plasmid Comprising of:

a nucleotide sequence encoding Methionine Aminopeptidase (MAP) protein or a variant thereof, operably linked to a promoter sequence of SEQ ID NO 5; and
a nucleotide sequence encoding protein of interest operably linked to a promoter sequence of SEQ ID NO 7;
   wherein the recombinant plasmid effects the protein of interest consisting of less than 2% N-terminal initiator methionine.

In another aspect of the present invention, the recombinant plasmid is a modified pET27b plasmid.

In another aspect of the present invention, the MAP protein or a variant thereof is further operably linked to a terminator.

In another aspect of the present invention, the protein of interest is further operably linked to a terminator.

In another aspect of the present invention, the nucleotide sequence encoding MAP protein operably linked to promoter sequence represented as SEQ ID NO: 2.

In another aspect of the present invention, the protein of interest is a protein of SEQ ID NO: 10.

In another aspect of the present invention, the protein of interest is a protein of SEQ ID NO: 13 or SEQ ID NO: 16.

In another aspect of the present invention, the MAP protein is a protein of SEQ ID NO: 4 or a variant thereof.

In another aspect of the present invention, the protein of interest can be used in cancer diagnosis or cancer therapy.

In another embodiment, the present invention relates to a use of recombinant plasmid to produce protein of interest with less than 2% N terminal initiator methionine.

In another embodiment of the present invention, the nucleotide sequence encoding protein of interest operably linked to a promoter is represented as FIG. 4.

In yet another aspect, the present invention relates to a recombinant plasmid of SEQ ID NO: 18, wherein the plasmid further comprises of a nucleotide sequence encoding protein of interest operably linked to a promoter sequence of SEQ ID NO: 7.

In yet another aspect, the present invention relates to a host cell comprising recombinant plasmid as claimed in claim 1, wherein the host cell is a prokaryote.

In yet another aspect of the present invention, the prokaryote is Escherichia coli DE3 strains.

In yet another aspect, the present invention relates to a method for producing protein of interest, comprising steps of:
a) providing a host cell with a recombinant plasmid as claimed in claim 1;
b) culturing the host cell in a culture medium;
c) purifying the protein of interest from the cultured host cell or the culture medium; and
   wherein the purified protein consists of less than 2% N terminal initiator methionine.

### In Another Embodiment, the Present Invention Relates to a Recombinant Plasmid Comprising of:

a nucleotide sequence of SEQ ID NO: 3 operably linked to a promoter sequence of SEQ ID NO: 5 and a terminator sequence; and
a nucleotide sequence encoding protein of interest operably linked to a promoter sequence of SEQ ID NO 7 and a terminator sequence;
wherein the recombinant plasmid effects the protein of interest consisting of less than 2% N terminal initiator methionine.

### BRIEF DESCRIPTION OF FIGURES

FIG. 1 represents DNA Construct comprising of TAC promoter, Methionine Aminopeptidase gene, and T3te terminator nucleotide sequence.

FIG. 2 depicts pET27b Plasmid.

FIG. 3 depicts pET27b Plasmid comprising of TAC promoter, Methionine Aminopeptidase gene, and T3te terminator.

FIG. 4 represents DNA Construct with Methionine Aminopeptidase gene along with gene expressing protein of interest.

FIG. 5 shows pET27b Plasmid construct having Methionine Aminopeptidase gene along with gene expressing protein of interest.

FIG. 6 represents Western Blot analysis of protein variant 1 and protein variant 2 co-expressed with Methionine aminopeptidase for reduced add-Met content along with respective controls.

### BRIEF DESCRIPTION OF SEQUENCE LISTING

SEQ ID NO: 1 represents DNA Construct of pET27b plasmid
atccggatatagttcctcctttcagcaaaaaacccctcaagacccgtttagaggccccaaggggt
tatgctagttattgctcagcggtggcagcagccaactcagcttcctttcgggctttgttagcagc
cggatctcagtggtggtggtggtggtgctcgacatcctcggggtcttccggggcgagttctggct
ggctagcccgtttgatctcgagtgcggccgcaagcttgtcgacggagctcgaattcggatccgaa
ttaattccgatatccatggccatcgccggctgggcagcgaggagcagcagaccagcagcagcggt
cggcagcaggtatttcatatgtatatctccttcttaaagttaaacaaaattatttctagagggga
attgttatccgctcacaattcccctatagtgagtcgtattaatttcgcgggatcgagatctcgat
cctctacgccggacgcatcgtggccggcatcaccggcgccacaggtgcggttgctggcgcctata
tcgccgacatcaccgatggggaagatcgggctcgccacttcgggctcatgagcgcttgtttcggc
gtgggtatggtggcaggccccgtggccgggggactgttgggcgccatctccttgcatgcaccatt
ccttgcggcggcggtgctcaacggcctcaacctactactgggctgcttcctaatgcaggagtcgc
ataagggagagcgtcgagatcccggacaccatcgaatggcgcaaaacctttcgcggtatggcatg
atagcgcccggaagagagtcaattcagggtggtgaatgtgaaaccagtaacgttatacgatgtcg
cagagtatgccggtgtctcttatcagaccgtttcccgcgtggtgaaccaggccagccacgtttct
gcgaaaacgcgggaaaaagtggaagcggcgatggcggagctgaattacattcccaaccgcgtggc
acaacaactggcgggcaaacagtcgttgctgattggcgttgccacctccagtctggccctgcacg
cgccgtcgcaaattgtcgcggcgattaaatctcgcgccgatcaactgggtgccagcgtggtggtg
tcgatggtagaacgaagcggcgtcgaagcctgtaaagcggcggtgcacaatcttctcgcgcaacg
cgtcagtgggctgatcattaactatccgctggatgaccaggatgccattgctgtggaagctgcct
gcactaatgttccggcgttatttcttgatgtctctgaccagacacccatcaacagtattattttc
tcccatgaagacggtacgcgactgggcgtggagcatctggtcgcattgggtcaccagcaaatcgc
gctgttagcgggcccattaagttctgtctcggcgcgtctgcgtctggctggctggcataaatatc
tcactcgcaatcaaattcagccgatagcggaacgggaaggcgactggagtgccatgtccggtttt
caacaaaccatgcaaatgctgaatgagggcatcgttcccactgcgatgctggttgccaacgatca
gatggcgctgggcgcaatgcgcgccattaccgagtccgggctgcgcgttggtgcggatatctcgg
tagtgggatacgacgataccgaagacagctcatgttatatcccgccgttaaccaccatcaaacag
gattttcgcctgctggggcaaaccagcgtggaccgcttgctgcaactctctcagggccaggcggt
gaagggcaatcagctgttgcccgtctcactggtgaaaagaaaaaccaccctggcgcccaatacgc
aaaccgcctctccccgcgcgttggccgattcattaatgcagctggcacgacaggtttcccgactg
gaaagcgggcagtgagcgcaacgcaattaatgtaagttagctcactcattaggcaccgggatctc
gaccgatgcccttgagagccttcaacccagtcagctccttccggtgggcgcggggcatgactatc
gtcgccgcacttatgactgtcttctttatcatgcaactcgtaggacaggtgccggcagcgctctg
ggtcattttcggcgaggaccgctttcgctggagcgcgacgatgatcggcctgtcgcttgcggtat
tcggaatcttgcacgccctcgctcaagccttcgtcactggtcccgccaccaaacgtttcggcgag
aagcaggccattatcgccggcatggcggccccacgggtgcgcatgatcgtgctcctgtcgttgag
gacccggctaggctggcggggttgccttactggttagcagaatgaatcaccgatacgcgagcgaa
cgtgaagcgactgctgctgcaaaacgtctgcgacctgagcaacaacatgaatggtcttcggtttc
cgtgtttcgtaaagtctggaaacgcggaagtcagcgccctgcaccattatgttccggatctgcat
cgcaggatgctgctggctaccctgtggaacacctacatctgtattaacgaagcgctggcattgac
cctgagtgatttttctctggtcccgccgcatccataccgccagttgtttaccctcacaacgttcc
agtaaccgggcatgttcatcatcagtaacccgtatcgtgagcatcctctctcgtttcatcggtat
cattacccccatgaacagaaatcccccttacacggaggcatcagtgaccaaacaggaaaaaaccg
cccttaacatggcccgctttatcagaagccagacattaacgcttctggagaaactcaacgagctg
gacgcggatgaacaggcagacatctgtgaatcgcttcacgaccacgctgatgagctttaccgcag
ctgcctcgcgcgtttcggtgatgacggtgaaaacctctgacacatgcagctcccggagacggtca
cagcttgtctgtaagcggatgccgggagcagacaagcccgtcagggcgcgtcagcgggtgttggc
gggtgtcggggcgcagccatgacccagtcacgtagcgatagcggagtgtatactggcttaactat
gcggcatcagagcagattgtactgagagtgcaccatatatgcggtgtgaaataccgcacagatgc
gtaaggagaaaataccgcatcaggcgctcttccgcttcctcgctcactgactcgctgcgctcggt
cgttcggctgcggcgagcggtatcagctcactcaaaggcggtaatacggttatccacagaatcag
gggataacgcaggaaagaacatgtgagcaaaaggccagcaaaaggccaggaaccgtaaaaaggcc
gcgttgctggcgtttttccataggctccgcccccctgacgagcatcacaaaaatcgacgctcaag
tcagaggtggcgaaacccgacaggactataaagataccaggcgtttccccctggaagctccctcg
tgcgctctcctgttccgaccctgccgcttaccggatacctgtccgcctttctcccttcgggaagc
gtggcgctttctcatagctcacgctgtaggtatctcagttcggtgtaggtcgttcgctccaagct
gggctgtgtgcacgaaccccccgttcagcccgaccgctgcgccttatccggtaactatcgtcttg
agtccaacccggtaagacacgacttatcgccactggcagcagccactggtaacaggattagcaga
gcgaggtatgtaggcggtgctacagagttcttgaagtggtggcctaactacggctacactagaag
gacagtatttggtatctgcgctctgctgaagccagttaccttcggaaaaagagttggtagctctt
gatccggcaaacaaaccaccgctggtagcggtggtttttttgtttgcaagcagcagattacgcgc
agaaaaaaaggatctcaagaagatcctttgatcttttctacggggtctgacgctcagtggaacga
aaactcacgttaagggattttggtcatgaacaataaaactgtctgcttacataaacagtaataca
aggggtgttatgagccatattcaacgggaaacgtcttgctctaggccgcgattaaattccaacat
ggatgctgatttatatgggtataaatgggctcgcgataatgtcgggcaatcaggtgcgacaatct
atcgattgtatgggaagcccgatgcgccagagttgtttctgaaacatggcaaaggtagcgttgcc
aatgatgttacagatgagatggtcagactaaactggctgacggaatttatgcctcttccgaccat
caagcattttatccgtactcctgatgatgcatggttactcaccactgcgatccccgggaaaacag
cattccaggtattagaagaatatcctgattcaggtgaaaatattgttgatgcgctggcagtgttc
ctgcgccggttgcattcgattcctgtttgtaattgtccttttaacagcgatcgcgtatttcgtct
cgctcaggcgcaatcacgaatgaataacggtttggttgatgcgagtgattttgatgacgagcgta
atggctggcctgttgaacaagtctggaaagaaatgcataaacttttgccattctcaccggattca
gtcgtcactcatggtgatttctcacttgataaccttatttttgacgaggggaaattaataggttg
tattgatgttggacgagtcggaatcgcagaccgataccaggatcttgccatcctatggaactgcc
tcggtgagttttctccttcattacagaaacggctttttcaaaaatatggtattgataatcctgat
atgaataaattgcagtttcatttgatgctcgatgagtttttctaagaattaattcatgagcggat
acatatttgaatgtatttagaaaaataaacaaataggggttccgcgcacatttccccgaaaagtg
ccacctgaaattgtaaacgttaatattttgttaaaattcgcgttaaatttttgttaaatcagctc
attttttaaccaataggccgaaatcggcaaaatcccttataaatcaaaagaatagaccgagatag
ggttgagtgttgttccagtttggaacaagagtccactattaaagaacgtggactccaacgtcaaa
gggcgaaaaaccgtctatcagggcgatggcccactacgtgaaccatcaccctaatcaagtttttt
ggggtcgaggtgccgtaaagcactaaatcggaaccctaaagggagcccccgatttagagcttgac
ggggaaagccggcgaacgtggcgagaaaggaagggaagaaagcgaaaggagcgggcgctagggcg
ctggcaagtgtagcggtcacgctgcgcgtaaccaccacacccgccgcgcttaatgcgccgctaca
gggcgcgtcccattcgcca
SEQ ID NO: 2 represents DNA Construct having TAC promoter,
Methionine Aminopeptidase (MAP) gene and T3te terminator
ttgacaattaatcatcggctcgtataatgtgtggaattgtgagcggataacaatttcacacagga
aacagccagtccgtttaggtgttttcacgagcacttcaccaacaaggaccatagactagtatggc
tatctcaatcaagaccccagaagatatcgaaaaaatgcgcgtcgctggccgactggctgccgaag
tgctggagatgatcgaaccgtatgttaaaccgggcgtcagcaccggcgagctggatcgcatctgt
aatgattacattgttaatgaacaacacgcggtttctgcctgcctcggctatcacggctatccgaa
atccgtttgcatctctattaatgaagtggtgtgccacggtatcccggacgatgctaagctgctga
aagatggcgatatcgttaacattgatgtcaccgtaatcaaagatggtttccacggcgatacctcg
aaaatgtttatgtcggtaagccgaccatcatgggcgaacgtctgtgccgcatcacgcaagaaagc
ctgtacctggcgctacgcatggtaaaaccaggcattaatctgcgcgaaatcggtgcggcgattca
gaaatttgtcgaagcagaaggcttctccgtcgttcgtgaatattgcggacacggtattggtcgcg
gcttccatgaagaaccgcaggtgctgcactatgactcccgtgaaaccaacgtcgtactgaaacct
gggatgacgttcaccatcgagccaatggtcaacgcgggtaaaaaagagatccgcaccatgaaaga
tggctggacggtaaaaaccaaagatcgcagcttgtctgcacaatatgagcatactattgtggtga
ctgataacggctgcgaaattctgacgctacgcaaggatgacaccatcccggcgataatctcgcac
gacgaataataggctcaccttcacgggtgggcctttcttcg
SEQ ID NO: 3 represents Nucleotide Sequence of Methionine
Aminopeptidase (MAP)
atggctatctcaatcaagaccccagaagatatcgaaaaaatgcgcgtcgctggccgactggctgc
cgaagtgctggagatgatcgaaccgtatgttaaaccgggcgtcagcaccggcgagctggatcgca
tctgtaatgattacattgttaatgaacaacacgcggtttctgcctgcctcggctatcacggctat
ccgaaatccgtttgcatctctattaatgaagtggtgtgccacggtatcccggacgatgctaagct
gctgaaagatggcgatatcgttaacattgatgtcaccgtaatcaaagatggtttccacggcgata
cctcgaaaatgtttatcgtcggtaagccgaccatcatgggcgaacgtctgtgccgcatcacgcaa
gaaagcctgtacctggcgctacgcatggtaaaaccaggcattaatctgcgcgaaatcggtgcggc
gattcagaaatttgtcgaagcagaaggcttctccgtcgttcgtgaatattgcggacacggtattg
gtcgcggcttccatgaagaaccgcaggtgctgcactatgactcccgtgaaaccaacgtcgtactg
aaacctgggatgacgttcaccatcgagccaatggtcaacgcgggtaaaaaagagatccgcaccat
gaaagatggctggacggtaaaaaccaaagatcgcagcttgtctgcacaatatgagcatactattg
tggtgactgataacggctgcgaaattctgacgctacgcaaggatgacaccatcccggcgataatc
tcgcacgacgaataata
SEQ ID NO: 4 represents Amino Acid Sequence of Methionine
Aminopeptidase (MAP)
MAISIKTPEDIEKMRVAGRLAAEVLEMIEPYVKPGVSTGELDRICNDYIVNEQHAVSACLGYHGY
PKSVCISINEVVCHGIPDDAKLLKDGDIVNIDVTVIKDGFHGDTSKMFIVGKPTIMGERLCRITQ
ESLYLALRMVKPGINLREIGAAIQKFVEAEGFSVVREYCGHGIGRGFHEEPQVLHYDSRETNVVL
KPGMTFTIEPMVNAGKKEIRTMKDGWTVKTKDRSLSAQYEHTIVVTDNGCEILTLRKDDTIPAII
SHDE
SEQ ID NO: 5 represents nucleotide sequence of TAC Promoter
ttgacaattaatcatcggctcgtataatg
SEQ ID NO: 6 represents nucleotide sequence of T3te terminator
ggctcaccttcacgggtgggcctttcttcg
SEQ ID NO: 7 represents nucleotide sequence of T7 promoter
taatacgactcactatagg
SEQ ID NO: 8 represents nucleotide sequence of T7 terminator
ctagcataaccccttggggcctctaaacgggtcttgaggggttttttg
SEQ ID NO: 9 represents nucleotide sequence of modified
Sclerotium rolfsii lectin
(Protein variant 1)
acctataaaattaccgtgcgcgtgtatcagaccaacccggatgcctttttccatccggtggaaaa
aaccgtgtggaaatatgcgaatggcggtacctggacgattacggatgatcagcatgtgctgacga
tgggtggtagcggtaccagcggcaccctgcgttttcacgcagataatggcgaaagcttcaccgcc
acctttggtgtgcataattataaacgctggtgtgatattgtgaccaacctggcagcggatgaaac
cggcatggttattaatcagcagtattatagtcagaaaaaccgcgaagaagcgcgtgaacgccagc
tgagtaactatcaggtgaaaaatgcgaaaggccgtaacttccagattgtttataccgaagcggaa
ggcaatgatctgcatgcgaacctgattatcggc
SEQ ID NO: 10 represents amino acid sequence of modified
Sclerotium rolfsii lectin (Protein variant 1)
TYKITVRVYQTNPDAFFHPVEKTVWKYANGGTWTITDDQHVLTMGGSGTSGTLRFHADNGESFTA
TFGVHNYKRWCDIVTNLAADETGMVINQQYYSQKNREEARERQLSNYQVKNAKGRNFQIVYTEAE
GNDLHANLIIG
SEQ ID NO: 11 represents nucleotide sequence of DNA construct
for expression of modified Sclerotium rolfsii lectin
(Protein variant 1)
taatacgactcactataggggaattgtgagcggataacaattcccctctagaaataattttgttt
aactttaagaaggagatatacatatgacctataaaattaccgtgcgcgtgtatcagaccaacccg
gatgcctttttccatccggtggaaaaaaccgtgtggaaatatgcgaatggcggtacctggacgat
tacggatgatcagcatgtgctgacgatgggtggtagcggtaccagcggcaccctgcgttttcacg
cagataatggcgaaagcttcaccgccacctttggtgtgcataattataaacgctggtgtgatatt
gtgaccaacctggcagcggatgaaaccggcatggttattaatcagcagtattatagtcagaaaaa
ccgcgaagaagcgcgtgaacgccagctgagtaactatcaggtgaaaaatgcgaaaggccgtaact
tccagattgtttataccgaagcggaaggcaatgatctgcatgcgaacctgattatcggctaatga
ggatccgaattcgagctccgtcgacaagcttgcggccgcactcgagatcaaacgggctagccagc
cagaactcgccccggaagaccccgaggatgtcgagcaccaccaccaccaccactgagatccggct
gctaacaaagcccgaaaggaagctgagttggctgctgccaccgctgagcaataactagcataacc
ccttggggcctctaaacgggtcttgaggggttttttg
SEQ ID NO: 12 represents nucleotide sequence of modified
Sclerotium rolfsii lectin
(Protein variant 2)
agctataaaattaccgtgcgcgtgtatcagaccaacccggatgcctttttccatccggtggaaaa
aaccgtgtggaaatatgcgaatggcggtacctggacgattacggatgatcagcatgtgctgacga
tgggtggtagcggtaccagcggcaccctgcgttttcacgcagataatggcgaaagcttcaccgcc
acctttggtgtgcataattataaacgctggggcgatattgtgaccaacctggcagcggatgaaac
cggcatggttattaatcagcagtattatagtcagaaaaaccgcgaagaagcgcgtgaacgccagc
tgagtaactatcaggtgaaaaatgcgaaaggccgtaacttccagattgtttataccgaagcggaa
ggcaatgatctgcatgcgaacctgattatcggctgc
SEQ ID NO: 13 represents amino acid sequence of modified
Sclerotium rolfsii lectin (Protein variant 2)
SYKITVRVYQTNPDAFFHPVEKTVWKYANGGTWTITDDQHVLTMGGSGTSGTLRFHADNGESFTA
TFGVHNYKRWGDIVTNLAADETGMVINQQYYSQKNREEARERQLSNYQVKNAKGRNFQIVYTEAE
GNDLHANLIIGC
SEQ ID NO: 14 represents nucleotide sequence of DNA construct for
expression of modified Sclerotium rolfsii lectin (Protein variant 2)
taatacgactcactataggggaattgtgagcggataacaattcccctctagaaataattttgttt
aactttaagaaggagatatacatatgagctataaaattaccgtgcgcgtgtatcagaccaacccg
gatgcctttttccatccggtggaaaaaaccgtgtggaaatatgcgaatggcggtacctggacgat
tacggatgatcagcatgtgctgacgatgggtggtagcggtaccagcggcaccctgcgttttcacg
cagataatggcgaaagcttcaccgccacctttggtgtgcataattataaacgctggggcgatatt
gtgaccaacctggcagcggatgaaaccggcatggttattaatcagcagtattatagtcagaaaaa
ccgcgaagaagcgcgtgaacgccagctgagtaactatcaggtgaaaaatgcgaaaggccgtaact
tccagattgtttataccgaagcggaaggcaatgatctgcatgcgaacctgattatcggctgctaa
tgaggatccgaattcgagctccgtcgacaagcttgcggccgcactcgagatcaaacgggctagcc
agccagaactcgccccggaagaccccgaggatgtcgagcaccaccaccaccaccactgagatccg
gctgctaacaaagcccgaaaggaagctgagttggctgctgccaccgctgagcaataactagcata
accccttggggcctctaaacgggtcttgaggggttttttg
SEQ ID NO: 15 represents nucleotide sequence of modified Sclerotium
rolfsii lectin (Protein variant 3)
agctataaaattaccgtgcgcgtgtatcagaccaacccggatgcctttttccatccggtggaaaa
aaccgtgtggaaatatgcgaatggcggtacctggacgattacggatgatcagcatgtgctgacga
tgggtggtagcggtaccagcggcaccctgcgttttcacgcagataatggcgaaagcttcaccgcc
acctttggtgtgcataattataaacgctggggcgatattgtgaccaacctggcagcggatgaaac
cggcatggttattaatcagcagtattatagtcagaaaaaccgcgaagaagcgcgtgaacgccagc
tgagtaactatcaggtgaaaaatgcgaaaggccgtaacttccagattgtttataccgaagcggaa
ggcaatgatctgcatgcgaacctgattatcggcagctgc
SEQ ID NO: 16 represents amino acid sequence of modified Sclerotium
rolfsii lectin (Protein variant 3)
SYKITVRVYQTNPDAFFHPVEKTVWKYANGGTWTITDDQHVLTMGGSGTSGTLRFHADNGESFTA
TFGVHNYKRWGDIVTNLAADETGMVINQQYYSQKNREEARERQLSNYQVKNAKGRNFQIVYTEAE
GNDLHANLIIGSC
SEQ ID NO: 17 represents nucleotide sequence of DNA construct for
expression of modified Sclerotium rolfsii lectin (Protein
variant 3)
taatacgactcactataggggaattgtgagcggataacaattcccctctagaaataattttgttt
aactttaagaaggagatatacatatgagctataaaattaccgtgcgcgtgtatcagaccaacccg
gatgcctttttccatccggtggaaaaaaccgtgtggaaatatgcgaatggcggtacctggacgat
tacggatgatcagcatgtgctgacgatgggtggtagcggtaccagcggcaccctgcgttttcacg
cagataatggcgaaagcttcaccgccacctttggtgtgcataattataaacgctggggcgatatt
gtgaccaacctggcagcggatgaaaccggcatggttattaatcagcagtattatagtcagaaaaa
ccgcgaagaagcgcgtgaacgccagctgagtaactatcaggtgaaaaatgcgaaaggccgtaact
tccagattgtttataccgaagcggaaggcaatgatctgcatgcgaacctgattatcggcagctgc
taatgaggatccgaattcgagctccgtcgacaagcttgcggccgcactcgagatcaaacgggcta
gccagccagaactcgccccggaagaccccgaggatgtcgagcaccaccaccaccaccactgagat
ccggctgctaacaaagcccgaaaggaagctgagttggctgctgccaccgctgagcaataactagc
ataaccccttggggcctctaaacgggtcttgaggggttttttg
SEQ ID NO: 18 represents nucleotide sequence of modified plasmid
with TAC promoter, Methionine Aminopeptidase (MAP) gene and
T3te terminator
atccggatatagttcctcctttcagcaaaaaacccctcaagacccgtttagaggccccaaggggt
tatgctagttattgctcagcggtggcagcagccaactcagcttcctttcgggctttgttagcagc
cggatctcagtggtggtggtggtggtgctcgacatcctcggggtcttccggggcgagttctggct
ggctagcccgtttgatctcgagtgcggccgcaagcttgtcgacggagctcgaattcggatccgaa
ttaattccgatatccatggccatcgccggctgggcagcgaggagcagcagaccagcagcagcggt
cggcagcaggtatttcatatgtatatctccttcttaaagttaaacaaaattatttctagagggga
attgttatccgctcacaattcccctatagtgagtcgtattaatttcgcgggatcgagatctggta
ccgagctgttgacaattaatcatcggctcgtataatgtgtggaattgtgagcggataacaatttc
acacaggaaacagccagtccgtttaggtgttttcacgagcacttcaccaacaaggaccatagact
agtatggctatctcaatcaagaccccagaagatatcgaaaaaatgcgcgtcgctggccgactggc
tgccgaagtgctggagatgatcgaaccgtatgttaaaccgggcgtcagcaccggcgagctggatc
gcatctgtaatgattacattgttaatgaacaacacgcggtttctgcctgcctcggctatcacggc
tatccgaaatccgtttgcatctctattaatgaagtggtgtgccacggtatcccggacgatgctaa
gctgctgaaagatggcgatatcgttaacattgatgtcaccgtaatcaaagatggtttccacggcg
atacctcgaaaatgtttatcgtcggtaagccgaccatcatgggcgaacgtctgtgccgcatcacg
caagaaagcctgtacctggcgctacgcatggtaaaaccaggcattaatctgcgcgaaatcggtgc
ggcgattcagaaatttgtcgaagcagaaggcttctccgtcgttcgtgaatattgcggacacggta
ttggtcgcggcttccatgaagaaccgcaggtgctgcactatgactcccgtgaaaccaacgtcgta
ctgaaacctgggatgacgttcaccatcgagccaatggtcaacgcgggtaaaaaagagatccgcac
catgaaagatggctggacggtaaaaaccaaagatcgcagcttgtctgcacaatatgagcatacta
ttgtggtgactgataacggctgcgaaattctgacgctacgcaaggatgacaccatcccggcgata
atctcgcacgacgaataataggctcaccttcacggggggcctttcttcggcatgcaccattcctt
gcggcggcggtgctcaacggcctcaacctactactgggctgcttcctaatgcaggagtcgcataa
gggagagcgtcgagatcccggacaccatcgaatggcgcaaaacctttcgcggtatggcatgatag
cgcccggaagagagtcaattcagggtggtgaatgtgaaaccagtaacgttatacgatgtcgcaga
gtatgccggtgtctcttatcagaccgtttcccgcgtggtgaaccaggccagccacgtttctgcga
aaacgcgggaaaaagtggaagcggcgatggcggagctgaattacattcccaaccgcgtggcacaa
caactggcgggcaaacagtcgttgctgattggcgttgccacctccagtctggccctgcacgcgcc
gtcgcaaattgtcgcggcgattaaatctcgcgccgatcaactgggtgccagcgtggtggtgtcga
tggtagaacgaagcggcgtcgaagcctgtaaagcggggtgcacaatcttctcgcgcaacgcgtca
gtgggctgatcattaactatccgctggatgaccaggatgccattgctgtggaagctgcctgcact
aatgttccggcgttatttcttgatgtctctgaccagacacccatcaacagtattattttctccca
tgaagacggtacgcgactgggcgtggagcatctggtcgcattgggtcaccagcaaatcgcgctgt
tagcgggcccattaagttctgtctcggcgcgtctgcgtctggctggctggcataaatatctcact
cgcaatcaaattcagccgatagcggaacgggaaggcgactggagtgccatgtccggttttcaaca
aaccatgcaaatgctgaatgagggcatcgttcccactgcgatgctggttgccaacgatcagatgg
cgctgggcgcaatgcgcgccattaccgagtccgggctgcgcgttggtgcggatatctcggtagtg
ggatacgacgataccgaagacagctcatgttatatcccgccgttaaccaccatcaaacaggattt
tcgcctgctggggcaaaccagcgtggaccgcttgctgcaactctctcagggccaggcggtgaagg
gcaatcagctgttgcccgtctcactggtgaaaagaaaaaccaccctggcgcccaatacgcaaacc
gcctctccccgcgcgttggccgattcattaatgcagctggcacgacaggtttcccgactggaaag
cgggcagtgagcgcaacgcaattaatgtaagttagctcactcattaggcaccgggatctcgaccg
atgcccttgagagccttcaacccagtcagctccttccggtgggcgcggggcatgactatcgtcgc
cgcacttatgactgtcttctttatcatgcaactcgtaggacaggtgccggcagcgctctgggtca
ttttcggcgaggaccgctttcgctggagcgcgacgatgatcggcctgtcgcttgcggtattcgga
atcttgcacgccctcgctcaagccttcgtcactggtcccgccaccaaacgtttcggcgagaagca
ggccattatcgccggcatggcggccccacgggtgcgcatgatcgtgctcctgtcgttgaggaccc
ggctaggctggcggggttgccttactggttagcagaatgaatcaccgatacgcgagcgaacgtga
agcgactgctgctgcaaaacgtctgcgacctgagcaacaacatgaatggtcttcggtttccgtgt
ttcgtaaagtctggaaacgcggaagtcagcgccctgcaccattatgttccggatctgcatcgcag
gatgctgctggctaccctgtggaacacctacatctgtattaacgaagcgctggcattgaccctga
gtgatttttctctggtcccgccgcatccataccgccagttgtttaccctcacaacgttccagtaa
ccgggcatgttcatcatcagtaacccgtatgtgagcatcctctctcgtttcatcggtatcattac
ccccatgaacagaaatcccccttacacggaggcatcagtgaccaaacaggaaaaaaccgccctta
acatggcccgctttatcagaagccagacattaacgcttctggagaaactcaacgagctggacgcg
gatgaacaggcagacatctgtgaatcgcttcacgaccacgctgatgagctttaccgcagctgcct
cgcgcgtttcggtgatgacggtgaaaacctctgacacatgcagctcccggagacggtcacagctt
gtctgtaagcggatgccgggagcagacaagcccgtcagggcgcgtcagcgggtgttggcgggtgt
cggggcgcagccatgacccagtcacgtagcgatagcggagtgtatactggcttaactatgcggca
tcagagcagattgtactgagagtgcaccatatatgcggtgtgaaataccgcacagatgcgtaagg
agaaaataccgcatcaggcgctcttccgcttcctcgctcactgactcgctgcgctcggtcgttcg
gctgcggcgagcggtatcagctcactcaaaggcggtaatacggttatccacagaatcaggggata
acgcaggaaagaacatgtgagcaaaaggccagcaaaaggccaggaaccgtaaaaaggccgcgttg
ctggcgtttttccataggctccgcccccctgacgagcatcacaaaaatcgacgctcaagtcagag
gtggcgaaacccgacaggactataaagataccaggcgtttccccctggaagctccctcgtgcgct
ctcctgttccgaccctgccgcttaccggatacctgtccgcctttctcccttcgggaagcgtggcg
ctttctcatagctcacgctgtaggtatctcagttcggtgtaggtcgttcgctccaagctgggctg
tgtgcacgaaccccccgttcagcccgaccgctgcgccttatccggtaactatcgtcttgagtcca
acccggtaagacacgacttatcgccactggcagcagccactggtaacaggattagcagagcgagg
tatgtaggcggtgctacagagttcttgaagtggtggcctaactacggctacactagaaggacagt
atttggtatctgcgctctgctgaagccagttaccttcggaaaaagagttggtagctcttgatccg
gcaaacaaaccaccgctggtagcggtggtttttttgtttgcaagcagcagattacgcgcagaaaa
aaaggatctcaagaagatcctttgatcttttctacggggtctgacgctcagtggaacgaaaactc
acgttaagggattttggtcatgaacaataaaactgtctgcttacataaacagtaatacaaggggt
gttatgagccatattcaacgggaaacgtcttgctctaggccgcgattaaattccaacatggatgc
tgatttatatgggtataaatgggctcgcgataatgtcgggcaatcaggtgcgacaatctatcgat
tgtatgggaagcccgatgcgccagagttgtttctgaaacatggcaaaggtagcgttgccaatgat
gttacagatgagatggtcagactaaactggctgacggaatttatgcctcttccgaccatcaagca
ttttatccgtactcctgatgatgcatggttactcaccactgcgatccccgggaaaacagcattcc
aggtattagaagaatatcctgattcaggtgaaaatattgttgatgcgctggcagtgttcctgcgc
cggttgcattcgattcctgtttgtaattgtccttttaacagcgatcgcgtatttcgtctcgctca
ggcgcaatcacgaatgaataacggtttggttgatgcgagtgattttgatgacgagcgtaatggct
ggcctgttgaacaagtctggaaagaaatgcataaacttttgccattctcaccggattcagtcgtc
actcatggtgatttctcacttgataaccttatttttgacgaggggaaattaataggttgtattga
tgttggacgagtcggaatcgcagaccgataccaggatcttgccatcctatggaactgcctcggtg
agttttctccttcattacagaaacggctttttcaaaaatatggtattgataatcctgatatgaat
aaattgcagtttcatttgatgctcgatgagtttttctaagaattaattcatgagcggatacatat
ttgaatgtatttagaaaaataaacaaataggggttccgcgcacatttccccgaaaagtgccacct
gaaattgtaaacgttaatattttgttaaaattcgcgttaaatttttgttaaatcagctcattttt
taaccaataggccgaaatcggcaaaatcccttataaatcaaaagaatagaccgagatagggttga
gtgttgttccagtttggaacaagagtccactattaaagaacgtggactccaacgtcaaagggcga
aaaaccgtctatcagggcgatggcccactacgtgaaccatcaccctaatcaagttttttggggtc
gaggtgccgtaaagcactaaatcggaaccctaaagggagcccccgatttagagcttgacggggaa
agccggcgaacgtggcgagaaaggaagggaagaaagcgaaaggagcgggcgctagggcgctggca
agtgtagcggtcacgctgcgcgtaaccaccacacccgccgcgcttaatgcgccgctacagggcgc
gtcccattcgcca
SEQ ID NO: 19 represents nucleotide sequence of plasmid with
modified Sclerotium rolfsii lectin (Protein variant 1) gene
atccggatatagttcctcctttcagcaaaaaacccctcaagacccgtttagaggccccaaggggt
tatgctagttattgctcagcggtggcagcagccaactcagcttcctttcgggctttgttagcagc
cggatctcagtggtggtggtggtggtgctcgacatcctcggggtcttccggggcgagttctggct
ggctagcccgtttgatctcgagtgcggccgcaagcttgtcgacggagctcgaattcggatcctca
ttagccgataatcaggttcgcatgcagatcattgccttccgcttcggtataaacaatctggaagt
tacggcctttcgcatttttcacctgatagttactcagctggcgttcacgcgcttcttcgcggttt
ttctgactataatactgctgattaataaccatgccggtttcatccgctgccaggttggtcacaat
atcacaccagcgtttataattatgcacaccaaaggtggcggtgaagctttcgccattatctgcgt
gaaaacgcagggtgccgctggtaccgctaccacccatcgtcagcacatgctgatcatccgtaatc
gtccaggtaccgccattcgcatatttccacacggttttttccaccggatggaaaaaggcatccgg
gttggtctgatacacgcgcacggtaattttataggtcatatgtatatctccttcttaaagttaaa
caaaattatttctagaggggaattgttatccgctcacaattcccctatagtgagtcgtattaatt
tcgcgggatcgagatctggtaccgagctgttgacaattaatcatcggctcgtataatgtgtggaa
ttgtgagcggataacaatttcacacaggaaacagccagtccgtttaggtgttttcacgagcactt
caccaacaaggaccatagactagtatggctatctcaatcaagaccccagaagatatcgaaaaaat
gcgcgtcgctggccgactggctgccgaagtgctggagatgatcgaaccgtatgttaaaccgggcg
tcagcaccggcgagctggatcgcatctgtaatgattacattgttaatgaacaacacgcggtttct
gcctgcctcggctatcacggctatccgaaatccgtttgcatctctattaatgaagtggtgtgcca
cggtatcccggacgatgctaagctgctgaaagatggcgatatcgttaacattgatgtcaccgtaa
tcaaagatggtttccacggcgatacctcgaaaatgtttatcgtcggtaagccgaccatcatgggc
gaacgtctgtgccgcatcacgcaagaaagcctgtacctggcgctacgcatggtaaaaccaggcat
taatctgcgcgaaatcggtgcggcgattcagaaatttgtcgaagcagaaggcttctccgtcgttc
gtgaatattgcggacacggtattggtcgcggcttccatgaagaaccgcaggtgctgcactatgac
tcccgtgaaaccaacgtcgtactgaaacctgggatgacgttcaccatcgagccaatggtcaacgc
gggtaaaaaagagatccgcaccatgaaagatggctggacggtaaaaaccaaagatcgcagcttgt
ctgcacaatatgagcatactattgtggtgactgataacggctgcgaaattctgacgctacgcaag
gatgacaccatcccggcgataatctcgcacgacgaataataggctcaccttcacggggggccttt
cttcggcatgcaccattccttgcggcggcggtgctcaacggcctcaacctactactgggctgctt
cctaatgcaggagtcgcataagggagagcgtcgagatcccggacaccatcgaatggcgcaaaacc
tttcgcggtatggcatgatagcgcccggaagagagtcaattcagggtggtgaatgtgaaaccagt
aacgttatacgatgtcgcagagtatgccggtgtctcttatcagaccgtttcccgcgtggtgaacc
aggccagccacgtttctgcgaaaacgcgggaaaaagtggaagcggcgatggcggagctgaattac
attcccaaccgcgtggcacaacaactggcgggcaaacagtcgttgctgattggcgttgccacctc
cagtctggccctgcacgcgccgtcgcaaattgtcgcggcgattaaatctcgcgccgatcaactgg
gtgccagcgtggtggtgtcgatggtagaacgaagcggcgtcgaagcctgtaaagcggcggtgcac
aatcttctcgcgcaacgcgtcagtgggctgatcattaactatccgctggatgaccaggatgccat
tgctgtggaagctgcctgcactaatgttccggcgttatttcttgatgtctctgaccagacaccca
tcaacagtattattttctcccatgaagacggtacgcgactgggcgtggagcatctggtcgcattg
ggtcaccagcaaatcgcgctgttagcgggcccattaagttctgtctcggcgcgtctgcgtctggc
tggctggcataaatatctcactcgcaatcaaattcagccgatagcggaacgggaaggcgactgga
gtgccatgtccggttttcaacaaaccatgcaaatgctgaatgagggcatcgttcccactgcgatg
ctggttgccaacgatcagatggcgctgggcgcaatgcgcgccattaccgagtccgggctgcgcgt
tggtgcggatatctcggtagtgggatacgacgataccgaagacagctcatgttatatcccgccgt
taaccaccatcaaacaggattttcgcctgctggggcaaaccagcgtggaccgcttgctgcaactc
tctcagggccaggcggtgaagggcaatcagctgttgcccgtctcactggtgaaaagaaaaaccac
cctggcgcccaatacgcaaaccgcctctccccgcgcgttggccgattcattaatgcagctggcac
gacaggtttcccgactggaaagcgggcagtgagcgcaacgcaattaatgtaagttagctcactca
ttaggcaccgggatctcgaccgatgcccttgagagccttcaacccagtcagctccttccggtggg
cgcggggcatgactatcgtcgccgcacttatgactgtcttctttatcatgcaactcgtaggacag
gtgccggcagcgctctgggtcattttcggcgaggaccgctttcgctggagcgcgacgatgatcgg
cctgtcgcttgcggtattcggaatcttgcacgccctcgctcaagccttcgtcactggtcccgcca
ccaaacgtttcggcgagaagcaggccattatcgccggcatggcggccccacgggtgcgcatgatc
gtgctcctgtcgttgaggacccggctaggctggcggggttgccttactggttagcagaatgaatc
accgatacgcgagcgaacgtgaagcgactgctgctgcaaaacgtctgcgacctgagcaacaacat
gaatggtcttcggtttccgtgtttcgtaaagtctggaaacgcggaagtcagcgccctgcaccatt
atgttccggatctgcatcgcaggatgctgctggctaccctgtggaacacctacatctgtattaac
gaagcgctggcattgaccctgagtgatttttctctggtcccgccgcatccataccgccagttgtt
taccctcacaacgttccagtaaccgggcatgttcatcatcagtaacccgtatcgtgagcatcctc
tctcgtttcatcggtatcattacccccatgaacagaaatcccccttacacggaggcatcagtgac
caaacaggaaaaaaccgcccttaacatggcccgctttatcagaagccagacattaacgcttctgg
agaaactcaacgagctggacgcggatgaacaggcagacatctgtgaatcgcttcacgaccacgct
gatgagctttaccgcagctgcctcgcgcgtttcggtgatgacggtgaaaacctctgacacatgca
gctcccggagacggtcacagcttgtctgtaagcggatgccgggagcagacaagcccgtcagggcg
cgtcagcgggtgttggcgggtgtcggggcgcagccatgacccagtcacgtagcgatagcggagtg
tatactggcttaactatgcggcatcagagcagattgtactgagagtgcaccatatatgcggtgtg
aaataccgcacagatgcgtaaggagaaaataccgcatcaggcgctcttccgcttcctcgctcact
gactcgctgcgctcggtcgttcggctgcggcgagcggtatcagctcactcaaaggcggtaatacg
gttatccacagaatcaggggataacgcaggaaagaacatgtgagcaaaaggccagcaaaaggcca
ggaaccgtaaaaaggccgcgttgctggcgtttttccataggctccgcccccctgacgagcatcac
aaaaatcgacgctcaagtcagaggtggcgaaacccgacaggactataaagataccaggcgtttcc
ccctggaagctccctcgtgcgctctcctgttccgaccctgccgcttaccggatacctgtccgcct
ttctcccttcgggaagcgtggcgctttctcatagctcacgctgtaggtatctcagttcggtgtag
gtcgttcgctccaagctgggctgtgtgcacgaaccccccgttcagcccgaccgctgcgccttatc
cggtaactatcgtcttgagtccaacccggtaagacacgacttatcgccactggcagcagccactg
gtaacaggattagcagagcgaggtatgtaggcggtgctacagagttcttgaagtggtggcctaac
tacggctacactagaaggacagtatttggtatctgcgctctgctgaagccagttaccttcggaaa
aagagttggtagctcttgatccggcaaacaaaccaccgctggtagcggtggtttttttgtttgca
agcagcagattacgcgcagaaaaaaaggatctcaagaagatcctttgatcttttctacggggtct
gacgctcagtggaacgaaaactcacgttaagggattttggtcatgaacaataaaactgtctgctt
acataaacagtaatacaaggggtgttatgagccatattcaacgggaaacgtcttgctctaggccg
cgattaaattccaacatggatgctgatttatatgggtataaatgggctcgcgataatgtcgggca
atcaggtgcgacaatctatcgattgtatgggaagcccgatgcgccagagttgtttctgaaacatg
gcaaaggtagcgttgccaatgatgttacagatgagatggtcagactaaactggctgacggaattt
atgcctcttccgaccatcaagcattttatccgtactcctgatgatgcatggttactcaccactgc
gatccccgggaaaacagcattccaggtattagaagaatatcctgattcaggtgaaaatattgttg
atgcgctggcagtgttcctgcgccggttgcattcgattcctgtttgtaattgtccttttaacagc
gatcgcgtatttcgtctcgctcaggcgcaatcacgaatgaataacggtttggttgatgcgagtga
ttttgatgacgagcgtaatggctggcctgttgaacaagtctggaaagaaatgcataaacttttgc
cattctcaccggattcagtcgtcactcatggtgatttctcacttgataaccttatttttgacgag
gggaaattaataggttgtattgatgttggacgagtcggaatcgcagaccgataccaggatcttgc
catcctatggaactgcctcggtgagttttctccttcattacagaaacggctttttcaaaaatatg
gtattgataatcctgatatgaataaattgcagtttcatttgatgctcgatgagtttttctaagaa
ttaattcatgagcggatacatatttgaatgtatttagaaaaataaacaaataggggttccgcgca
catttccccgaaaagtgccacctgaaattgtaaacgttaatattttgttaaaattcgcgttaaat
ttttgttaaatcagctcattttttaaccaataggccgaaatcggcaaaatcccttataaatcaaa
agaatagaccgagatagggttgagtgttgttccagtttggaacaagagtccactattaaagaacg
tggactccaacgtcaaagggcgaaaaaccgtctatcagggcgatggcccactacgtgaaccatca
ccctaatcaagttttttggggtcgaggtgccgtaaagcactaaatcggaaccctaaagggagccc
ccgatttagagcttgacggggaaagccggcgaacgtggcgagaaaggaagggaagaaagcgaaag
gagcgggcgctagggcgctggcaagtgtagcggtcacgctgcgcgtaaccaccacacccgccgcg
cttaatgcgccgctacagggcgcgtcccattcgcca
SEQ ID NO: 20 represents nucleotide sequence of plasmid with
modified Sclerotium rolfsii lectin (Protein variant 2) gene
atccggatatagttcctcctttcagcaaaaaacccctcaagacccgtttagaggccccaaggggt
tatgctagttattgctcagcggtggcagcagccaactcagcttcctttcgggctttgttagcagc
cggatctcagtggtggtggtggtggtgctcgacatcctcggggtcttccggggcgagttctggct
ggctagcccgtttgatctcgagtgcggccgcaagcttgtcgacggagctcgaattcggatcctca
ttagcagccgataatcaggttcgcatgcagatcattgccttccgcttcggtataaacaatctgga
agttacggcctttcgcatttttcacctgatagttactcagctggcgttcacgcgcttcttcgcgg
tttttctgactataatactgctgattaataaccatgccggtttcatccgctgccaggttggtcac
aatatcgccccagcgtttataattatgcacaccaaaggtggcggtgaagctttcgccattatctg
cgtgaaaacgcagggtgccgctggtaccgctaccacccatcgtcagcacatgctgatcatccgta
atcgtccaggtaccgccattcgcatatttccacacggttttttccaccggatggaaaaaggcatc
cgggttggtctgatacacgcgcacggtaattttatagctcatatgtatatctccttcttaaagtt
aaacaaaattatttctagaggggaattgttatccgctcacaattcccctatagtgagtcgtatta
atttcgcgggatcgagatctggtaccgagctgttgacaattaatcatcggctcgtataatgtgtg
gaattgtgagcggataacaatttcacacaggaaacagccagtccgtttaggtgttttcacgagca
cttcaccaacaaggaccatagactagtatggctatctcaatcaagaccccagaagatatcgaaaa
aatgcgcgtcgctggccgactggctgccgaagtgctggagatgatcgaaccgtatgttaaaccgg
gcgtcagcaccggcgagctggatcgcatctgtaatgattacattgttaatgaacaacacgcggtt
tctgcctgcctcggctatcacggctatccgaaatccgtttgcatctctattaatgaagtggtgtg
ccacggtatcccggacgatgctaagctgctgaaagatggcgatatcgttaacattgatgtcaccg
taatcaaagatggtttccacggcgatacctcgaaaatgtttatcgtcggtaagccgaccatcatg
ggcgaacgtctgtgccgcatcacgcaagaaagcctgtacctggcgctacgcatggtaaaaccagg
cattaatctgcgcgaaatcggtgcggcgattcagaaatttgtcgaagcagaaggcttctccgtcg
ttcgtgaatattgcggacacggtattggtcgcggcttccatgaagaaccgcaggtgctgcactat
gactcccgtgaaaccaacgtcgtactgaaacctgggatgacgttcaccatcgagccaatggtcaa
cgcgggtaaaaaagagatccgcaccatgaaagatggctggacggtaaaaaccaaagatcgcagct
tgtctgcacaatatgagcatactattgtggtgactgataacggctgcgaaattctgacgctacgc
aaggatgacaccatcccggcgataatctcgcacgacgaataataggctcaccttcacggggggcc
tttcttcggcatgcaccattccttgcggcggcggtgctcaacggcctcaacctactactgggctg
cttcctaatgcaggagtcgcataagggagagcgtcgagatcccggacaccatcgaatggcgcaaa
acctttcgcggtatggcatgatagcgcccggaagagagtcaattcagggtggtgaatgtgaaacc
agtaacgttatacgatgtcgcagagtatgccggtgtctcttatcagaccgtttcccgcgtggtga
accaggccagccacgtttctgcgaaaacgcgggaaaaagtggaagcggcgatggcggagctgaat
tacattcccaaccgcgtggcacaacaactggcgggcaaacagtcgttgctgattggcgttgccac
ctccagtctggccctgcacgcgccgtcgcaaattgtcgcggcgattaaatctcgcgccgatcaac
tgggtgccagcgtggtggtgtcgatggtagaacgaagcggcgtcgaagcctgtaaagcggcggtg
cacaatcttctcgcgcaacgcgtcagtgggctgatcattaactatccgctggatgaccaggatgc
cattgctgtggaagctgcctgcactaatgttccggcgttatttcttgatgtctctgaccagacac
ccatcaacagtattattttctcccatgaagacggtacgcgactgggcgtggagcatctggtcgca
ttgggtcaccagcaaatcgcgctgttagcgggcccattaagttctgtctcggcgcgtctgcgtct
ggctggctggcataaatatctcactcgcaatcaaattcagccgatagcggaacgggaaggcgact
ggagtgccatgtccggttttcaacaaaccatgcaaatgctgaatgagggcatcgttcccactgcg
atgctggttgccaacgatcagatggcgctgggcgcaatgcgcgccattaccgagtccgggctgcg
cgttggtgcggatatctcggtagtgggatacgacgataccgaagacagctcatgttatatcccgc
cgttaaccaccatcaaacaggattttcgcctgctggggcaaaccagcgtggaccgcttgctgcaa
ctctctcagggccaggcggtgaagggcaatcagctgttgcccgtctcactggtgaaaagaaaaac
caccctggcgcccaatacgcaaaccgcctctccccgcgcgttggccgattcattaatgcagctgg
cacgacaggtttcccgactggaaagcgggcagtgagcgcaacgcaattaatgtaagttagctcac
tcattaggcaccgggatctcgaccgatgcccttgagagccttcaacccagtcagctccttccggt
gggcgcggggcatgactatcgtcgccgcacttatgactgtcttctttatcatgcaactcgtagga
caggtgccggcagcgctctgggtcattttcggcgaggaccgctttcgctggagcgcgacgatgat
cggcctgtcgcttgcggtattcggaatcttgcacgccctcgctcaagccttcgtcactggtcccg
ccaccaaacgtttcggcgagaagcaggccattatcgccggcatggcggccccacgggtgcgcatg
atcgtgctcctgtcgttgaggacccggctaggctggcggggttgccttactggttagcagaatga
atcaccgatacgcgagcgaacgtgaagcgactgctgctgcaaaacgtctgcgacctgagcaacaa
catgaatggtcttcggtttccgtgtttcgtaaagtctggaaacgcggaagtcagcgccctgcacc
attatgttccggatctgcatcgcaggatgctgctggctaccctgtggaacacctacatctgtatt
aacgaagcgctggcattgaccctgagtgatttttctctggtcccgccgcatccataccgccagtt
gtttaccctcacaacgttccagtaaccgggcatgttcatcatcagtaacccgtatcgtgagcatc
ctctctcgtttcatcggtatcattacccccatgaacagaaatcccccttacacggaggcatcagt
gaccaaacaggaaaaaaccgcccttaacatggcccgctttatcagaagccagacattaacgcttc
tggagaaactcaacgagctggacgcggatgaacaggcagacatctgtgaatcgcttcacgaccac
gctgatgagctttaccgcagctgcctcgcgcgtttcggtgatgacggtgaaaacctctgacacat
gcagctcccggagacggtcacagcttgtctgtaagcggatgccgggagcagacaagcccgtcagg
gcgcgtcagcgggtgttggcgggtgtcggggcgcagccatgacccagtcacgtagcgatagcgga
gtgtatactggcttaactatgcggcatcagagcagattgtactgagagtgcaccatatatgcggt
gtgaaataccgcacagatgcgtaaggagaaaataccgcatcaggcgctcttccgcttcctcgctc
actgactcgctgcgctcggtcgttcggctgcggcgagcggtatcagctcactcaaaggcggtaat
acggttatccacagaatcaggggataacgcaggaaagaacatgtgagcaaaaggccagcaaaagg
ccaggaaccgtaaaaaggccgcgttgctggcgtttttccataggctccgcccccctgacgagcat
cacaaaaatcgacgctcaagtcagaggtggcgaaacccgacaggactataaagataccaggcgtt
tccccctggaagctccctcgtgcgctctcctgttccgaccctgccgcttaccggatacctgtccg
cctttctcccttcgggaagcgtggcgctttctcatagctcacgctgtaggtatctcagttcggtg
taggtcgttcgctccaagctgggctgtgtgcacgaaccccccgttcagcccgaccgctgcgcctt
atccggtaactatcgtcttgagtccaacccggtaagacacgacttatcgccactggcagcagcca
ctggtaacaggattagcagagcgaggtatgtaggcggtgctacagagttcttgaagtggtggcct
aactacggctacactagaaggacagtatttggtatctgcgctctgctgaagccagttaccttcgg
aaaaagagttggtagctcttgatccggcaaacaaaccaccgctggtagcggtggtttttttgttt
gcaagcagcagattacgcgcagaaaaaaaggatctcaagaagatcctttgatcttttctacgggg
tctgacgctcagtggaacgaaaactcacgttaagggattttggtcatgaacaataaaactgtctg
cttacataaacagtaatacaaggggtgttatgagccatattcaacgggaaacgtcttgctctagg
ccgcgattaaattccaacatggatgctgatttatatgggtataaatgggctcgcgataatgtcgg
gcaatcaggtgcgacaatctatcgattgtatgggaagcccgatgcgccagagttgtttctgaaac
atggcaaaggtagcgttgccaatgatgttacagatgagatggtcagactaaactggctgacggaa
tttatgcctcttccgaccatcaagcattttatccgtactcctgatgatgcatggttactcaccac
tgcgatccccgggaaaacagcattccaggtattagaagaatatcctgattcaggtgaaaatattg
ttgatgcgctggcagtgttcctgcgccggttgcattcgattcctgtttgtaattgtccttttaac
agcgatcgcgtatttcgtctcgctcaggcgcaatcacgaatgaataacggtttggttgatgcgag
tgattttgatgacgagcgtaatggctggcctgttgaacaagtctggaaagaaatgcataaacttt
tgccattctcaccggattcagtcgtcactcatggtgatttctcacttgataaccttatttttgac
gaggggaaattaataggttgtattgatgttggacgagtcggaatcgcagaccgataccaggatct
tgccatcctatggaactgcctcggtgagttttctccttcattacagaaacggctttttcaaaaat
atggtattgataatcctgatatgaataaattgcagtttcatttgatgctcgatgagtttttctaa
gaattaattcatgagcggatacatatttgaatgtatttagaaaaataaacaaataggggttccgc
gcacatttccccgaaaagtgccacctgaaattgtaaacgttaatattttgttaaaattcgcgtta
aatttttgttaaatcagctcattttttaaccaataggccgaaatcggcaaaatcccttataaatc
aaaagaatagaccgagatagggttgagtgttgttccagtttggaacaagagtccactattaaaga
acgtggactccaacgtcaaagggcgaaaaaccgtctatcagggcgatggcccactacgtgaacca
tcaccctaatcaagttttttggggtcgaggtgccgtaaagcactaaatcggaaccctaaagggag
cccccgatttagagcttgacggggaaagccggcgaacgtggcgagaaaggaagggaagaaagcga
aaggagcgggcgctagggcgctggcaagtgtagcggtcacgctgcgcgtaaccaccacacccgcc
gcgcttaatgcgccgctacagggcgcgtcccattcgcca
SEQ ID NO: 21 represents nucleotide sequence of plasmid with
modified Sclerotium rolfsii lectin (Protein variant 3) gene
atccggatatagttcctcctttcagcaaaaaacccctcaagacccgtttagaggccccaaggggt
tatgctagttattgctcagcggtggcagcagccaactcagcttcctttcgggctttgttagcagc
cggatctcagtggtggtggtggtggtgctcgacatcctcggggtcttccggggcgagttctggct
ggctagcccgtttgatctcgagtgcggccgcaagcttgtcgacggagctcgaattcggatcctca
ttagcagctgccgataatcaggttcgcatgcagatcattgccttccgcttcggtataaacaatct
ggaagttacggcctttcgcatttttcacctgatagttactcagctggcgttcacgcgcttcttcg
cggtttttctgactataatactgctgattaataaccatgccggtttcatccgctgccaggttggt
cacaatatcgccccagcgtttataattatgcacaccaaaggtggcggtgaagctttcgccattat
ctgcgtgaaaacgcagggtgccgctggtaccgctaccacccatcgtcagcacatgctgatcatcc
gtaatcgtccaggtaccgccattcgcatatttccacacggttttttccaccggatggaaaaaggc
atccgggttggtctgatacacgcgcacggtaattttatagctcatatgtatatctccttcttaaa
gttaaacaaaattatttctagaggggaattgttatccgctcacaattcccctatagtgagtcgta
ttaatttcgcgggatcgagatctggtaccgagctgttgacaattaatcatcggctcgtataatgt
gtggaattgtgagcggataacaatttcacacaggaaacagccagtccgtttaggtgttttcacga
gcacttcaccaacaaggaccatagactagtatggctatctcaatcaagaccccagaagatatcga
aaaaatgcgcgtcgctggccgactggctgccgaagtgctggagatgatcgaaccgtatgttaaac
cgggcgtcagcaccggcgagctggatcgcatctgtaatgattacattgttaatgaacaacacgcg
gtttctgcctgcctcggctatcacggctatccgaaatccgtttgcatctctattaatgaagtggt
gtgccacggtatcccggacgatgctaagctgctgaaagatggcgatatcgttaacattgatgtca
ccgtaatcaaagatggtttccacggcgatacctcgaaaatgtttatcgtcggtaagccgaccatc
atgggcgaacgtctgtgccgcatcacgcaagaaagcctgtacctggcgctacgcatggtaaaacc
aggcattaatctgcgcgaaatcggtgcggcgattcagaaatttgtcgaagcagaaggcttctccg
tcgttcgtgaatattgcggacacggtattggtcgcggcttccatgaagaaccgcaggtgctgcac
tatgactcccgtgaaaccaacgtcgtactgaaacctgggatgacgttcaccatcgagccaatggt
caacgcgggtaaaaaagagatccgcaccatgaaagatggctggacggtaaaaaccaaagatcgca
gcttgtctgcacaatatgagcatactattgtggtgactgataacggctgcgaaattctgacgcta
cgcaaggatgacaccatcccggcgataatctcgcacgacgaataataggctcaccttcacggggg
gcctttcttcggcatgcaccattccttgcggcggcggtgctcaacggcctcaacctactactggg
ctgcttcctaatgcaggagtcgcataagggagagcgtcgagatcccggacaccatcgaatggcgc
aaaacctttcgcggtatggcatgatagcgcccggaagagagtcaattcagggtggtgaatgtgaa
accagtaacgttatacgatgtcgcagagtatgccggtgtctcttatcagaccgtttcccgcgtgg
tgaaccaggccagccacgtttctgcgaaaacgcgggaaaaagtggaagcggcgatggcggagctg
aattacattcccaaccgcgtggcacaacaactggcgggcaaacagtcgttgctgattggcgttgc
cacctccagtctggccctgcacgcgccgtcgcaaattgtcgcggcgattaaatctcgcgccgatc
aactgggtgccagcgtggtggtgtcgatggtagaacgaagcggcgtcgaagcctgtaaagcggcg
gtgcacaatcttctcgcgcaacgcgtcagtgggctgatcattaactatccgctggatgaccagga
tgccattgctgtggaagctgcctgcactaatgttccggcgttatttcttgatgtctctgaccaga
cacccatcaacagtattattttctcccatgaagacggtacgcgactgggcgtggagcatctggtc
gcattgggtcaccagcaaatcgcgctgttagcgggcccattaagttctgtctcggcgcgtctgcg
tctggctggctggcataaatatctcactcgcaatcaaattcagccgatagcggaacgggaaggcg
actggagtgccatgtccggttttcaacaaaccatgcaaatgctgaatgagggcatcgttcccact
gcgatgctggttgccaacgatcagatggcgctgggcgcaatgcgcgccattaccgagtccgggct
gcgcgttggtgcggatatctcggtagtgggatacgacgataccgaagacagctcatgttatatcc
cgccgttaaccaccatcaaacaggattttcgcctgctggggcaaaccagcgtggaccgcttgctg
caactctctcagggccaggcggtgaagggcaatcagctgttgcccgtctcactggtgaaaagaaa
aaccaccctggcgcccaatacgcaaaccgcctctccccgcgcgttggccgattcattaatgcagc
tggcacgacaggtttcccgactggaaagcgggcagtgagcgcaacgcaattaatgtaagttagct
cactcattaggcaccgggatctcgaccgatgcccttgagagccttcaacccagtcagctccttcc
ggtgggcgcggggcatgactatcgtcgccgcacttatgactgtcttctttatcatgcaactcgta
ggacaggtgccggcagcgctctgggtcattttcggcgaggaccgctttcgctggagcgcgacgat
gatcggcctgtcgcttgcggtattcggaatcttgcacgccctcgctcaagccttcgtcactggtc
ccgccaccaaacgtttcggcgagaagcaggccattatcgccggcatggcggccccacgggtgcgc
atgatgtgctcctgtcgttgaggacccggctaggctggcggggttgccttactggttagcagaat
gaatcaccgatacgcgagcgaacgtgaagcgactgctgctgcaaaacgtctgcgacctgagcaac
aacatgaatggtcttcggtttccgtgtttcgtaaagtctggaaacgcggaagtcagcgccctgca
ccattatgttccggatctgcatcgcaggatgctgctggctaccctgtggaacacctacatctgta
ttaacgaagcgctggcattgaccctgagtgatttttctctggtcccgccgcatccataccgccag
ttgtttaccctcacaacgttccagtaaccgggcatgttcatcatcagtaacccgtatcgtgagca
tcctctctcgtttcatcggtatcattacccccatgaacagaaatcccccttacacggaggcatca
gtgaccaaacaggaaaaaaccgcccttaacatggcccgctttatcagaagccagacattaacgct
tctggagaaactcaacgagctggacgcggatgaacaggcagacatctgtgaatcgcttcacgacc
acgctgatgagctttaccgcagctgcctcgcgcgtttcggtgatgacggtgaaaacctctgacac
atgcagctcccggagacggtcacagcttgtctgtaagcggatgccgggagcagacaagcccgtca
gggcgcgtcagcgggtgttggcgggtgtcggggcgcagccatgacccagtcacgtagcgatagcg
gagtgtatactggcttaactatgcggcatcagagcagattgtactgagagtgcaccatatatgcg
gtgtgaaataccgcacagatgcgtaaggagaaaataccgcatcaggcgctcttccgcttcctcgc
tcactgactcgctgcgctcggtcgttcggctgcggcgagcggtatcagctcactcaaaggcggta
atacggttatccacagaatcaggggataacgcaggaaagaacatgtgagcaaaaggccagcaaaa
ggccaggaaccgtaaaaaggccgcgttgctggcgtttttccataggctccgcccccctgacgagc
atcacaaaaatcgacgctcaagtcagaggtggcgaaacccgacaggactataaagataccaggcg
tttccccctggaagctccctcgtgcgctctcctgttccgaccctgccgcttaccggatacctgtc
cgcctttctcccttcgggaagcgtggcgctttctcatagctcacgctgtaggtatctcagttcgg
tgtaggtcgttcgctccaagctgggctgtgtgcacgaaccccccgttcagcccgaccgctgcgcc
ttatccggtaactatcgtcttgagtccaacccggtaagacacgacttatcgccactggcagcagc
cactggtaacaggattagcagagcgaggtatgtaggcggtgctacagagttcttgaagtggtggc
ctaactacggctacactagaaggacagtatttggtatctgcgctctgctgaagccagttaccttc
ggaaaaagagttggtagctcttgatccggcaaacaaaccaccgctggtagcggtggtttttttgt
ttgcaagcagcagattacgcgcagaaaaaaaggatctcaagaagatcctttgatcttttctacgg
ggtctgacgctcagtggaacgaaaactcacgttaagggattttggtcatgaacaataaaactgtc
tgcttacataaacagtaatacaaggggtgttatgagccatattcaacgggaaacgtcttgctcta
ggccgcgattaaattccaacatggatgctgatttatatgggtataaatgggctcgcgataatgtc
gggcaatcaggtgcgacaatctatcgattgtatgggaagcccgatgcgccagagttgtttctgaa
acatggcaaaggtagcgttgccaatgatgttacagatgagatggtcagactaaactggctgacgg
aatttatgcctcttccgaccatcaagcattttatccgtactcctgatgatgcatggttactcacc
actgcgatccccgggaaaacagcattccaggtattagaagaatatcctgattcaggtgaaaatat
tgttgatgcgctggcagtgttcctgcgccggttgcattcgattcctgtttgtaattgtcctttta
acagcgatcgcgtatttcgtctcgctcaggcgcaatcacgaatgaataacggtttggttgatgcg
agtgattttgatgacgagcgtaatggctggcctgttgaacaagtctggaaagaaatgcataaact
tttgccattctcaccggattcagtcgtcactcatggtgatttctcacttgataaccttatttttg
acgaggggaaattaataggttgtattgatgttggacgagtcggaatcgcagaccgataccaggat
cttgccatcctatggaactgcctcggtgagttttctccttcattacagaaacggctttttcaaaa
atatggtattgataatcctgatatgaataaattgcagtttcatttgatgctcgatgagtttttct
aagaattaattcatgagcggatacatatttgaatgtatttagaaaaataaacaaataggggttcc
gcgcacatttccccgaaaagtgccacctgaaattgtaaacgttaatattttgttaaaattcgcgt
taaatttttgttaaatcagctcattttttaaccaataggccgaaatcggcaaaatcccttataaa
tcaaaagaatagaccgagatagggttgagtgttgttccagtttggaacaagagtccactattaaa
gaacgtggactccaacgtcaaagggcgaaaaaccgtctatcagggcgatggcccactacgtgaac
catcaccctaatcaagttttttggggtcgaggtgccgtaaagcactaaatcggaaccctaaaggg
agcccccgatttagagcttgacggggaaagccggcgaacgtggcgagaaaggaagggaagaaagc
gaaaggagcgggcgctagggcgctggcaagtgtagcggtcacgctgcgcgtaaccaccacacccg
ccgcgcttaatgcgccgctacagggcgcgtcccattcgcca

### DETAILED DESCRIPTION OF THE INVENTION

### Definitions

As used herein, the term “comprising” shall be understood as referring to an open definition, allowing further members of similar or other features.

As used herein, the term “consists of” or “consisting of” shall be understood as a closed definition relating to a limited range of features.

As used herein, the term ‘Methionine aminopeptidases’ or MAP are metalloenzymes that cleave the N-terminal methionine from newly synthesized peptides and proteins. MAP initiate co- and post-translational modifications that are essential for the translocation, activation, regulation, and degradation of proteins. The MAP as used herein may be an enzyme expressed in native form or may be genetically engineered to suit specific needs.

As used herein, the term ‘N terminal initiator methionine’ or ‘N terminal methionine’ refers to a Methionine amino acid present at the amino terminal end of the expressed protein. The said methionine is later co-translationally cleaved by enzyme methionine aminopeptidase (MAP) to form a mature protein.

As used herein, the term ‘DNA construct’ or ‘gene construct’ refers to an artificially designed segment of DNA borne on a vector that can be used to incorporate genetic material into a target tissue or cell. The words ‘DNA construct’ or ‘gene construct’ may be used interchangeably and shall construe the same meaning. ‘DNA construct’ or ‘gene construct’ comprises of a promoter sequence, open reading frame sequence and the terminator sequence. The said construct may further comprise of special sequences such as an enhancer, silencer, or reporter sequence depending on the nature of protein to be expressed.

As used herein, a ‘Host cell’ is a cell used for expression of protein of interest. Host cell can be modified by transformation of a vector enabling expression of protein of interest. Non limiting examples of host cell includes plant, animal, human, bacteria, yeast, or filamentous fungi cells.

As used herein, the expression ‘protein with minimal N-terminal initiator methionine’ refers to protein comprising equal to or not more than 2% N terminal initiator methionine.

As used herein, the term ‘promoter’ refers to a region of DNA where RNA polymerase begins to transcribe a gene. Promoter sequences are typically located directly upstream or at the 5′ end of the transcription initiation site. The promoter also functions as a regulatory region for gene expression.

As used herein, the term ‘terminator’ refers to a region of DNA at the end of a gene or operon, which causes transcription to stop. Terminator sequences follow the promoter and coding region and is typically located downstream or at the 3′ end of the gene.

As used herein, the term “pET plasmid” is low copy number plasmid, thereby reducing the leaky expression before induction. Target genes are cloned in pET plasmids under control of strong bacteriophage T7 transcription and (optionally) translation signals, wherein expression is induced by providing a source of T7 RNA polymerase in the host cell.

As used herein, the term ‘Open Reading Frame (ORF)’ refers to portion of a DNA sequence that spans between the start and stop codon. In other words, ORF is nucleotide sequence that can be translated into a polypeptide.

As used herein, the term ‘operably linked to’ refers to sequence having activity of the disclosure in a functional relationship with another nucleotide component of the nucleic acid molecule.

As used herein, the term “lectin” refers to carbohydrate-binding proteins that are highly specific to sugar groups that are part of other molecules, causing agglutination of particular cells or precipitation of glycoconjugates and polysaccharides.

As used herein, the term “Sclerotium rolfsii” is used to denote a soil borne phytopathogenic fungus from whose sclerotic bodies, lectin can be purified.

As used herein the phrase “the recombinant plasmid effects the protein of interest” refers to the use of the recombinant plasmid of present invention in the method of preparation of protein of interest which resulted in the protein of interest consisting of less than 2% N-terminal initiator methionine.

### Source of Biological Material:

Biological material used in the present disclosure is Escherichia coli strain BL21 DE3 and has been sourced commercially from Stratagene (An Agilent Technologies Div.), USA.

Embodiments of the present disclosure are not intended to limit the scope of disclosure to that which is illustrated and disclosed in this description.

Embodiments are provided to thoroughly and fully convey the scope of the present disclosure to the person skilled in the art. Numerous details are set forth, relating to specific components, and processes, to provide a complete understanding of embodiments of the present disclosure. It will be apparent to the person skilled in the art that the details provided in the embodiments should not be construed to limit the scope of the present disclosure. In some embodiments, well-known composition, well-known processes, and well-known techniques are not described in detail.

It is understood that each feature or embodiment, or combination, described herein is a non-limiting, illustrative example of any of the aspects of the disclosure and, as such, is meant to be combinable with any other feature or embodiment, or combination, described herein. For example, where features are described with language such as “embodiment”, “one of the embodiments”, “one more embodiment”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the disclosure.

The removal of the N-terminal initiator methionine is a critical step in the maturation of expressed proteins. Protein synthesis is initiated by either methionine in eukaryotes or formylmethionine in prokaryotes. Methionine aminopeptidases (MAP) are metalloenzymes that cleave the N-terminal methionine from newly synthesized peptides and proteins. MAP initiate co- and post-translational modifications that are essential for the translocation, activation, regulation, and degradation of proteins. These MAP enzymes are present in prokaryotes, yeast, and eukaryotes. The methionine aminopeptidases isolated from prokaryotes are MAP1 while those isolated from eukaryotes are MAP2. As per reports, the host cell without exogenous or additional copies of MAP gene expresses recombinant protein with N terminal methionine at a concentration between 10%-90% of total expressed protein.

In an aspect of the present disclosure, a plasmid is designed and constructed for expression of protein with minimal N-terminal initiator methionine.

In an embodiment of the present disclosure, a DNA construct comprising of a promoter region, gene/ORF, and terminator/stop region is designed. The said construct may further comprise of special sequences such as an enhancer, silencer, or reporter sequence depending on the nature of protein to be expressed.

In an embodiment, the present invention relates to a recombinant plasmid comprising of:
a nucleotide sequence encoding Methionine Aminopeptidase (MAP) protein or a variant thereof, operably linked to a promoter sequence of SEQ ID NO 5; and
a nucleotide sequence encoding protein of interest operably linked to a promoter sequence of SEQ ID NO 7;
   wherein the recombinant plasmid effects the protein of interest consisting of less than 2% N-terminal initiator methionine.

In another embodiment, the present invention relates to a recombinant plasmid comprising of:
a nucleotide sequence of SEQ ID NO: 3 operably linked to a promoter sequence of SEQ ID NO: 5 and a terminator sequence; and
a nucleotide sequence encoding protein of interest operably linked to a promoter sequence of SEQ ID NO 7 and a terminator sequence;
   wherein the recombinant plasmid effects the protein of interest consisting of less than 2% N terminal initiator methionine.

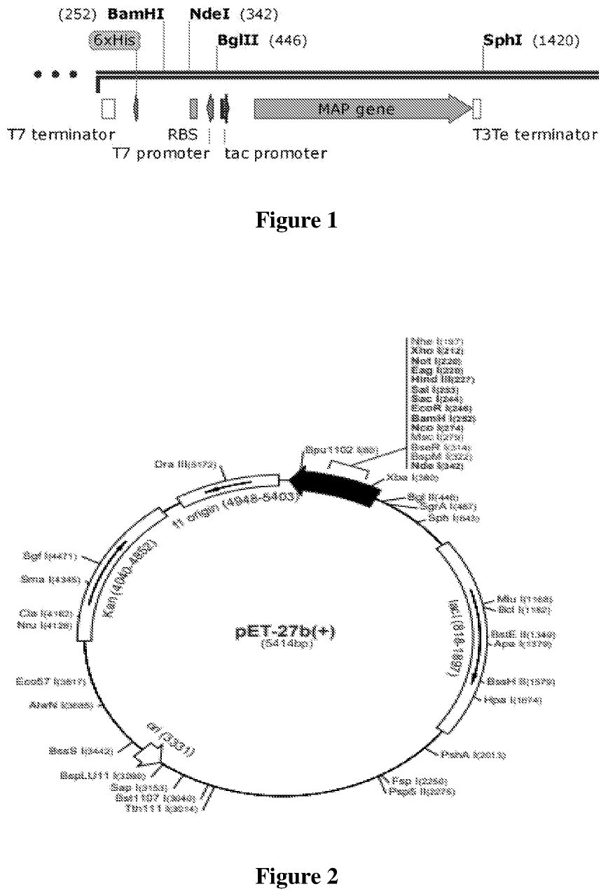

In one of the embodiments of the present disclosure, a DNA construct comprising of a gene expressing methionine aminopeptidase (MAP) is designed. The said gene is flanked by a promoter sequence at 5′ end and a terminator sequence at 3′ end of the Open Reading Frame (ORF) of methionine aminopeptidase gene. In one more embodiment of the present disclosure, DNA construct comprising of promoter, Methionine aminopeptidase (MAP) gene and terminator sequence designed is as shown in FIG. 1. It is further envisaged that sequence used for expression of methionine aminopeptidase (MAP) may comprise of a native (wild) sequence or a modified sequence. It is further envisaged that the said MAP may also be replaced with an enzyme having similar activity as that of MAP.

In one of the embodiments of the present disclosure, promoter operably linked to a MAP gene is selected in such a manner as to provide optimal expression of a MAP, and further MAP activity results in protein with minimal N terminal initiator methionine. It is very well understood by the person skilled in the art that selection of promoter may be made in such a manner that it does not affect the yield of the expressed protein of interest.

In one of the embodiments of the present disclosure, promoters selected from araE, UV5 and/or TAC (pTac) are evaluated for expression of protein with minimal N terminal initiator methionine by operably linking promoter to MAP gene. In one more embodiment of the present disclosure, the promoter sequence operably linked to a MAP gene is a TAC promoter, more particularly having nucleic acid sequence as represented by SEQ ID NO: 5.

In one of the embodiments of the present disclosure, terminator sequence operably linked to a MAP gene is selected in such a manner as to provide optimal expression of a MAP, and MAP activity further results in protein expression with minimal N terminal initiator methionine. It is very well understood by the person skilled in the art that selection of terminator sequence may be made in such a manner that it does not affect the yield of the expressed protein. In one more embodiment of the present disclosure, the terminator sequence operably linked to a MAP gene is a T3te terminator, more particularly having nucleic acid sequence as represented by SEQ ID NO: 6.

In one of the embodiments of the present disclosure, a DNA construct comprising of a gene expressing methionine aminopeptidase (MAP) comprises of a TAC promoter sequence as specified in SEQ ID NO: 5; Methionine aminopeptidase (MAP) gene as specified in SEQ ID NO: 3; and T3te terminator sequence as specified by SEQ ID NO: 6. In one more embodiment, the DNA construct prepared is represented as SEQ ID NO: 2.

In an embodiment of the present disclosure, a DNA construct comprising of a gene expressing methionine aminopeptidase (MAP) is cloned into a suitable vector or plasmid. Vector or plasmid suitable of expression of recombinant proteins in a host cell may be selected based on the suitability of expression of recombinant proteins in host cell. The suitable vector may also be selected on the basis of copy number of said vector or plasmid, wherein copy number enable efficient expression of the protein of interest as well as MAP protein. The expression vector may be selected from pGEX and pET series of vectors. The pET series plasmids are a low copy number plasmids, thereby reducing the leaky expression before induction. Target genes are cloned in pET plasmids under control of strong bacteriophage T7 transcription and (optionally) translation signals, wherein expression is induced by providing a source of T7 RNA polymerase in the host cell.

It is envisaged that the pET series plasmids comprise of one or more key components as mentioned below:
T7 promoter: Drives high-level transcription of the gene of interest when T7 RNA polymerase is present. When placed immediately upstream of a LacO element, the entire cassette is known as the T7lac promoter.
LacO: Binding site for LacI. This element inhibits activity of the T7 promoter when LacI protein is present, preventing leaky expression of the gene of interest.
RBS: The ribosome-binding site and translation initiation element from T7 bacteriophage. This allows for efficient production of the protein of interest.
ORF: The open reading frame of your gene of interest is placed here.
T7 terminator: Signal sequence to terminate the transcript made from the gene of interest, preventing run-on transcription.
Ampicillin: Ampicillin resistance gene. It allows the plasmid to be maintained by ampicillin selection in E. coli.
pBR322 ori: pBR322 origin of replication. Plasmids carrying this origin as well as the Rop gene exist in low copy numbers in E. coli.
Rop: Repressor of primer. It encodes a small protein that regulates plasmid copy number. The presence of the Rop protein, in combination of pBR322 origin of replication on the plasmid, results in low copy numbers of the plasmid.
LacI: The E. coli natural promoter and coding sequence for the lac repressor. In the absence of induction of the system (i.e., without IPTG), the LacI protein represses transcription of the gene of interest from the T7lac promoter, as well as transcription of T7 RNA polymerase from the LacUV5 promoter in host strains used for recombinant protein production.

In one of the embodiments of the present disclosure, a DNA construct comprising of a gene expressing methionine aminopeptidase (MAP) is cloned into a pET plasmid. In one more embodiment, the said modified pET plasmid comprises of DNA construct represented by SEQ ID NO: 2.

In one of the embodiments of the present disclosure, a DNA construct comprising of a gene expressing methionine aminopeptidase (MAP) is cloned into a pET plasmid. In one of the embodiments, the said DNA construct is cloned into the pET27b plasmid between restriction site BglII and SphI. In one more embodiment, the said modified pET27b plasmid comprising of DNA construct (SEQ ID NO: 2) is represented as SEQ ID NO: 18.

In one of the embodiments of the present disclosure, a DNA construct comprising of a gene expressing protein of interest (hereinafter referred as protein) is designed. The said gene is flanked by a promoter sequence at 5′ end and a terminator sequence at 3′ end of the Open Reading Frame (ORF) of gene encoding protein. In one more embodiment of the present disclosure, DNA construct comprising of promoter, gene encoding protein and terminator sequence designed is as shown in FIG. 4. It is further envisaged that gene sequence used for expression of protein may comprise of gene sequence encoding protein as desired by the person skilled in the art. It is further envisaged that the protein expressed may be selected from the group comprising of antibodies, contractile proteins, enzymes, hormonal proteins, structural proteins, storage proteins, and transport proteins.

In one of the embodiments of the present disclosure, promoter operably linked to a gene encoding protein is selected in such a manner as to provide optimal expression of a protein with minimal N-terminal initiator methionine. It is very well understood by the person skilled in the art that selection of promoter may be made in such a manner that it does not affect the yield of the expressed protein.

In one more embodiment of the present disclosure, the promoter sequence operably linked to a gene encoding protein is a T7 promoter, more particularly having nucleic acid sequence as represented by SEQ ID NO: 7.

In one more embodiment of the present disclosure, the terminator sequence operably linked to a gene encoding protein is a T7 terminator, more particularly having nucleic acid sequence as represented by SEQ ID NO: 8.

In one of the embodiments of the present disclosure, DNA construct comprising of a gene encoding protein comprises of a T7 promoter sequence as specified in SEQ ID NO: 7; gene encoding protein of interest; and T7 terminator sequence as specified by SEQ ID NO: 8. In one more embodiment, the DNA construct prepared is represented as FIG. 4. In one more embodiment of the present disclosure, gene encoding protein of interest is selected from SEQ ID NO: 9, SEQ ID NO: 12 & SEQ ID NO: 15.

In one of the embodiments of the present disclosure, a DNA construct comprising of a gene encoding protein as represented as FIG. 4 is one or more selected from SEQ ID NO: 11, SEQ ID NO: 14 & SEQ ID NO: 17.

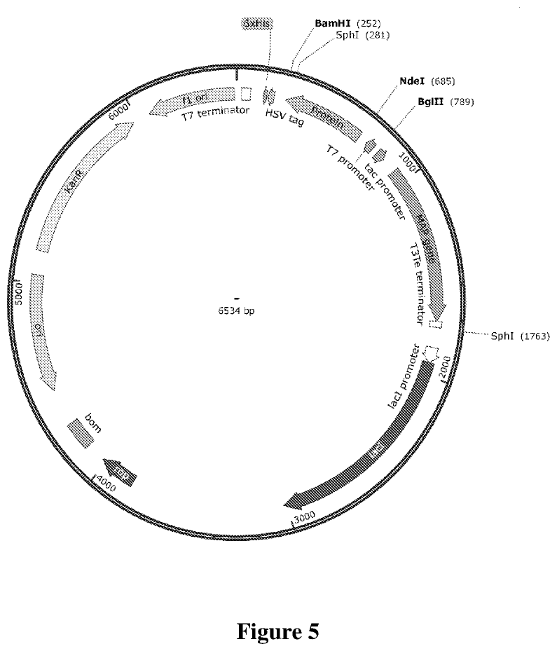

In one of the embodiments of the present disclosure, nucleotide sequence encoding a protein was cloned into modified pET27b plasmid as shown in FIG. 5. It is envisaged that said nucleotide sequence can be any sequence designed by the skilled person based on the protein to be expressed. In one more embodiment of the present disclosure, nucleotide Sequence expressing a protein is cloned in the MCS region between restrictions sites NdeI and BamHI of antisense strand of modified plasmid.

In one of the embodiments of the present disclosure, Nucleotide Sequence (SEQ ID NO: 9) encoding a protein of SEQ ID NO: 10 (Protein Variant 1) was cloned in the MCS region between restrictions sites NdeI and BamHI of antisense strand of modified plasmid (SEQ ID NO: 18). In one more embodiment of the present disclosure, modified plasmid having nucleotide sequence encoding a protein (Protein Variant 1) is SEQ ID NO: 19.

In one of the embodiments of the present disclosure, Nucleotide Sequence (SEQ ID NO: 12) encoding protein of SEQ ID NO: 13 (Protein Variant 2) was cloned in the MCS region between restrictions sites NdeI and BamHI of antisense strand of modified plasmid (SEQ ID NO: 18). In one more embodiment of the present disclosure, modified plasmid having nucleotide sequence encoding a protein (Protein Variant 2) is SEQ ID NO: 20.

In one more embodiment of the present disclosure, Nucleotide Sequence (SEQ ID NO: 15) encoding protein of SEQ ID NO: 16 (Protein Variant 3) was cloned in the MCS region between restrictions sites NdeI and BamHI of antisense strand of modified plasmid (SEQ ID NO: 18). In one more embodiment of the present disclosure, modified plasmid having nucleotide sequence encoding a protein (Protein Variant 3) is SEQ ID NO: 21.

In another aspect of the present disclosure, there is provided a method of expressing a protein of interest with minimal or reduced N terminal initiator methionine.

In yet another aspect, the present invention relates to a method for producing protein of interest, comprising steps of
a) providing a host cell with a recombinant plasmid as claimed in claim 1;
b) culturing the host cell in a culture medium;
c) purifying the protein of interest from the cultured host cell or the culture medium; and
   wherein the purified protein consists of less than 2% N terminal initiator methionine.

In an embodiment of the present disclosure, a method of expressing a protein with minimal or reduced N terminal initiator methionine comprises of constructing a vector having a nucleic acid sequence encoding a protein and a nucleic acid sequence encoding MAP (Methionine aminopeptidase); introducing the said vector into a host cell, co-expressing the gene of interest and MAP by the host cell such that the expressed MAP optimally cleaves the N-terminal methionine of said protein. The said expressed protein is further subjected to downstream purification and evaluation of purity and quality.

In one of the embodiments of the present disclosure, modified plasmid having nucleotide sequence encoding a protein is transformed into suitable host-cell by any of the known methods in art. In one more embodiment, the said modified plasmid having nucleotide sequence encoding a protein is transformed into host-cell by heat shock method. Selection of the transformed cells from the non-transformed population may be done by using selectable marker genes that confers resistance to antibiotics, and further clones with desired results selected for protein expression and analysis. The said host cell may be selected from the prokaryotes and eukaryotes, comprising of copies of the T7 RNA polymerase gene driven by the LacUV5 promoter. Expression of the T7 polymerase is induced by the addition of the lactose analog IPTG to the bacterial culture. The host cell may be selected from the group of prokaryotes comprising of Corynebacterium, Bacillus, Caulobacter, Phototrophic bacteria, Cold adapted bacteria, Halophilic bacteria, Streptomycetes, Nocardia, Mycobacteria, Lactic acid bacteria, and Escherichia coli, and the like. In one more embodiment of the present disclosure, host cell is a prokaryote, more specifically E coli. In a preferred embodiment, the Escherichia coli strain is having an inducible T7lac promoter (DE3) system can be used for protein production with construct or plasmid of the present disclosure. The E coli strain may be selected from BL21 (DE3) and its derivatives Rosetta2 (DE3), BL21(DE3) CodonPlus RP/RIL, Tuner(DE3), Bl21(DE3) Star, BL21-AI, BL21-SI, C41(DE3), C43(DE3); redox modified strains such as AD494(DE3), BL21(DE3) trxB, Origami (DE3), OrigamiB(DE3); and rec-strains such as BLR(DE3), JM109(DE3), HMS174(DE3).

In an embodiment of the present disclosure, the transformed host cells expressing protein were subjected to fermentation process. The said fermentation process may comprise of a suitable fermentation medium known to a person skilled in the art. The said media may be prepared inhouse or may be sourced from commercially available media. The fermentation culture is further envisaged to comprise induction of protein expression by introduction of an inducer Isopropyl β-D thiogalactopyranoside (IPTG). The fermentation broth is harvested upon start of decline phase of the fermentation growth curve and subjected to downstream purification process. It is envisaged that the downstream purification process may be designed by skilled person on the basis of nature of protein to be purified. The downstream process may comprise of centrifugation, chromatography, filtration, dialysis, and combination thereof.

In an embodiment of the present disclosure, expressed protein upon purification may be subjected to qualitative and quantitative analysis. It is envisaged that qualitative and quantitative analysis may be carried out by a skilled person using any suitable methods reported in the art. The protein may be analysed qualitatively and quantitatively for estimation of yield of protein, purity of the protein and n-terminal initiator methionine analysis. The protein analysis may include but not limited to use of reverse phase liquid chromatography (RP-HPLC), Liquid chromatography-mass spectrometry (LC-MS), High resolution mass spectrometry (HR-MS), Isoelectric Focusing, Capillary Isoelectric Focusing (cIEF), SDS-polyacrylamide gel electrophoresis, immune blotting, and the like.

In another aspect of the present disclosure, there is provided a protein with not more than 2% N terminal initiator methionine content.

In an embodiment of the present disclosure, protein is envisaged to be a recombinant protein with therapeutic, industrial and/or economic value. In one more embodiment of the present disclosure, the protein with not more than 2% N terminal initiator methionine content is a lectin protein. Lectins are a unique group of proteins (or glycoproteins) with potent biological activity and are important group of bioactive proteins found in most organisms and have received widespread attention for many years in the field of medicine as diagnostics and therapeutics. To date, lectins from various sources including plants, algae, fungi, and cyanobacteria have been isolated, characterised and found to differ in terms of physico-chemical characteristics like molecular size and sugar specificities. Plant lectins are found in wheat, corn, tomatoes, peanuts, kidney beans, bananas, peas, lentils, soybeans, mushrooms, tubers, seeds, mistletoe, and potatoes among many others. Similarly, Fungal lectins have been isolated from but not limited to Mycelium, Conidia, and Sclerotium Rolfsii, and the like.

In one more embodiment of the present disclosure, protein with not more than 2% N terminal initiator methionine content is a wildtype or modified lectin of Sclerotium rolfsii. Sclerotium rolfsii lectin (SRL) is a lectin isolated from fungus Sclerotium rolfsii and has high binding specificity towards the oncofetal Thomsen-Friedenreich carbohydrate antigen (Galβ1-3GalNAc-α-O-Ser/Thr, T or TF), which is expressed in more than 90% of human cancers. This specificity to TF antigen is being highly explored for its potential in onco-diagnostics and onco-therapeutics. As disclosed in EP2430041, a modified lectin protein with 141 amino acids have been expressed in E coli as stable and soluble protein using recombinant DNA technology. The said modified lectin is similar to the native lectin with regards to the sugar binding specificity and the apoptotic properties enabling them to be used as drug delivery agents for cancer treatment and application as diagnostic tools.

In one more embodiment of the present disclosure, protein with not more than 2% N terminal initiator methionine content is a wildtype or modified lectin of Sclerotium rolfsii. The said lectin expressed and purified as per aforementioned embodiments of this disclosure comprises of a protein selected from SEQ ID NO: 10, SEQ ID NO: 13 and SEQ ID NO: 16; wherein the said protein upon purification consists of not more than 2% N terminal initiator methionine.

While considerable emphasis has been placed herein on the specific features of the preferred embodiment, it will be appreciated that many additional features can be added and that many changes can be made in the preferred embodiment without departing from the principles of the disclosure. These and other changes in the preferred embodiment of the disclosure will be apparent to those skilled in the art from the disclosure herein, whereby it is to be distinctly understood that the foregoing descriptive matter is to be interpreted merely as illustration of the disclosure and not as a limitation.

### EXAMPLES

The following examples are included to demonstrate preferred embodiments of the disclosure. It should be appreciated by those of skill in the art that the compositions and techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: DNA Construct with MAP Gene

A DNA construct comprising of promoter, Methionine aminopeptidase (MAP) gene and terminator sequence was designed as shown in FIG. 1.

The said construct comprises of a TAC promoter sequence as specified in SEQ ID NO: 5; Methionine aminopeptidase (MAP) gene as specified in SEQ ID NO: 3; and T3te terminator sequence as specified by SEQ ID NO: 6.

The DNA construct prepared is represented as SEQ ID NO: 2.

### Example 2: Cloning of DNA Construct of SEQ ID NO: 2 into pET27b Plasmid

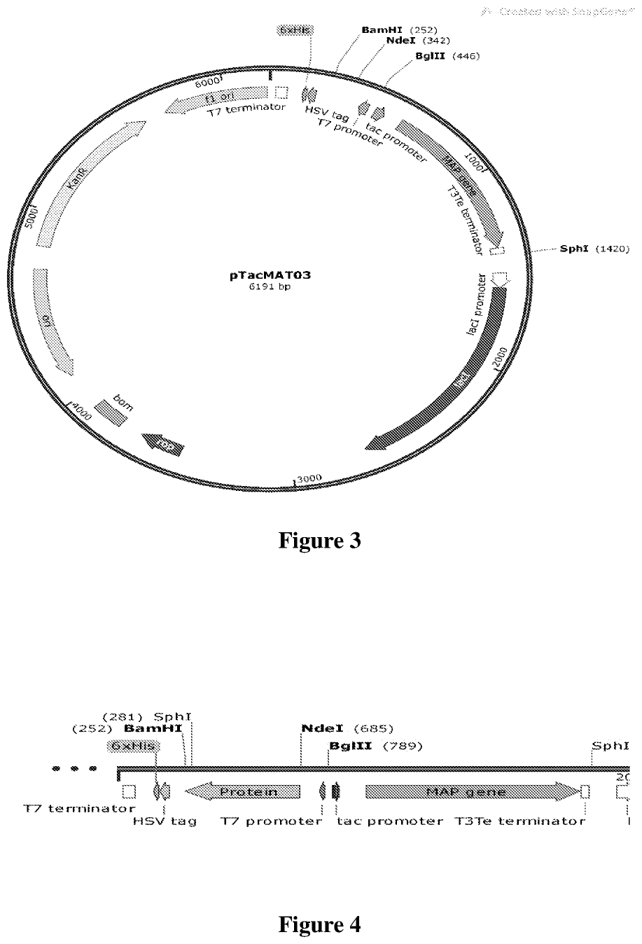

Cloning of DNA construct of SEQ ID NO: 2 into pET27b plasmid was done as shown in FIG. 3.

DNA construct of SEQ ID NO: 2 was cloned into the pET27b plasmid at restriction sites between BglII and SphI.

The modified pET27b plasmid comprising of DNA construct (SEQ ID NO: 2) is represented as SEQ ID NO: 18.

### Example 3: DNA Construct with Nucleotide Sequence of Protein to be Expressed

A DNA construct comprising of a promoter, nucleotide sequence of protein to be expressed, and terminator was designed as shown in FIG. 4.

Nucleotide Sequence (SEQ ID NO: 9) expressing a recombinant protein of SEQ ID NO: 10 was used in a DNA construct.

The SEQ ID NO: 9 is amplified with gene specific forward and reverse primers containing NdeI and BamHI sites, respectively, which was restriction digested and used to clone in respective sites at the multiple cloning site of the plasmid of SEQ ID NO: 18.

The said DNA construct comprised of a T7 promoter sequence as specified in SEQ ID NO: 7; Nucleic acid sequence of SEQ ID NO: 9, and T7 terminator sequence as specified in SEQ ID NO: 8. The said DNA construct is represented as SEQ ID NO: 11.

Similarly, individual DNA construct comprising nucleotide sequence of SEQ ID NO: 12 and SEQ ID NO: 15 was prepared. The said DNA construct were represented as SEQ ID NO: 14 and SEQ ID NO: 17.

### Example 4: Cloning of SEQ ID NO: 9 into Modified Plasmid of SEQ ID NO: 18

Cloning of nucleotide sequence expressing recombinant protein into modified pET27b plasmid was done as shown in FIG. 5.

Nucleotide Sequence (SEQ ID NO: 9) expressing a recombinant protein of SEQ ID NO: 10 was cloned in the MCS region between restrictions sites NdeI and BamHI of antisense strand of modified plasmid (SEQ ID NO: 18).

Similarly, individual modified plasmids comprising nucleotide sequence of SEQ ID NO: 12 and SEQ ID NO: 15 were prepared.

### Example 5: Transformation of E coli with Vector

Each of the modified plasmid as prepared in example 4 were transformed into E. coli cells by heat shock method as follows:

To 100 μl of competent E. coli BL21 DE3 cells, 100 ng of plasmid DNA was added and incubated on ice for 30 minutes. This transformation mixture was then subjected to heat shock at 42° C. for 90 seconds in a dry bath/water bath. 1 ml of Luria broth was added to transformation mix gently and incubated at 37° C.±2° C. for 60 minutes. 100 μl of the transformation mix was plated on to Luria Agar plates containing appropriate antibiotic and incubated overnight at 37° C.±2° C. Selected colonies were then sub-cultured into production media for assessment of protein expression. The clones with desired results were selected for further analysis.

### Example 6: Expression and Purification of Expressed Protein

Transformed E coli BL21 DE3 cell were used for expression of protein specified in SEQ ID NO: 10, SEQ ID NO: 13, SEQ ID NO: 16, using below mentioned process.

Inoculum medium used comprised of 1.5% Yeast Extract (w/v), 0.75% Na₂HPO₄2H₂O (w/v), 0.5% Dextrose (w/v), 0.1% MgSO₄·7H₂O (w/v), 0.5% NaCl (w/v), Kanamycin to a final concentration of 20 μg/ml and 0.1% (v/v) trace metal solution.

Fermentation medium comprised of 1% Yeast Extract (w/v), 0.3% KH₂PO₄ (w/v), 1.25% K₂HPO₄ (w/v), 0.5% (NH₄)₂SO₄ (w/v), 1.2% Dextrose (w/v), 0.1% MgSO₄·7H₂O (w/v), Kanamycin to a final concentration of 20 μg/ml, 0.1% (v/v) trace metal solution.

Transformed E coli BL21 DE3 cell were inoculated in inoculation medium under continuous shaking at 30° C. for 15 hours at 110 rpm. The 600 ml of inoculum was then transferred to a 6 liters of fermentation media at 14.7 OD at 600 nm. Fermentation process was carried out at 18° C. at air flow rates of 0.5-2.0 VVM throughout the process.

Dissolved Oxygen was maintained to ~60% with agitation and aeration with oxygen inputs. Foaming during the fermentation process was controlled by antifoaming agents. Agitation was maintained between 400-950 RPM. Fermentation pH was maintained at 6.8±0.6 using 10N sodium hydroxide solution. Feeding was initiated with glycerol as carbon source and yeast extract as nitrogen source at 5 hours. Fermentation culture was induced with 0.25 mM Isopropyl β-D thiogalactopyranoside (IPTG) for protein expression at 10 hours. Harvesting was done at 48 hours/OD 105 at 600 nm.

The harvest broth was centrifuged to obtain pellets. The pellets were subjected to chromatography techniques. The purification involved passing through 4 columns. Column 1 was a Cellufine Max Q-r Ion Exchange Chromatography column. The eluate was further passed through a Hydrophobic Interaction Chromatography column. The resulting eluate was passed through an SP Sepharose Ion Exchange Chromatography column. The last column was Ion Exchange Chromatography column Source 30 Q.

Purified proteins were further analysed for quantitative and qualitative analysis.

### Example 7: Analysis of Expressed Protein

Purified protein obtained in example 6 were analyzed for yield of expressed protein and the purity contents.

Expressed protein of SEQ ID NO: 10 in E coli without exogenous MAP gene expression system was used as control for analysis purpose.

The results obtained have been tabulated in TABLE NO: 1

Western blot analysis of protein of SEQ ID NO: 10 co-expressed with Methionine aminopeptidase and SEQ ID NO: 16 co-expressed with Methionine aminopeptidase was done to check the expression of Methionine aminopeptidase by the pET27b plasmid. The respective controls were pET27b plasmids without additional Methionine aminopeptidase gene.
ULLB-0005 (−) MAP Uninduced-SEQ ID 10 without MAP gene (Control)
ULLB-0005 (−) MAP Induced-SEQ ID 10 without MAP gene (Control with induction)
ULLB-1411-0011 (−) MAP Uninduced-SEQ ID 16 without MAP gene (Control)
ULLB-1411-0011 (−) MAP Induced-SEQ ID 16 without MAP gene (Control with
induction)
ULLB-0005 (+) MAP Uninduced-SEQ ID 10 with MAP gene without induction
ULLB-0005 (+) MAP Induced-SEQ ID 10 with MAP gene with induction
ULLB-1411-0011 (+) MAP Uninduced-SEQ ID 16 with MAP gene without induction
ULLB-1411-0011 (+) MAP Induced-SEQ ID 16 with MAP gene with induction)

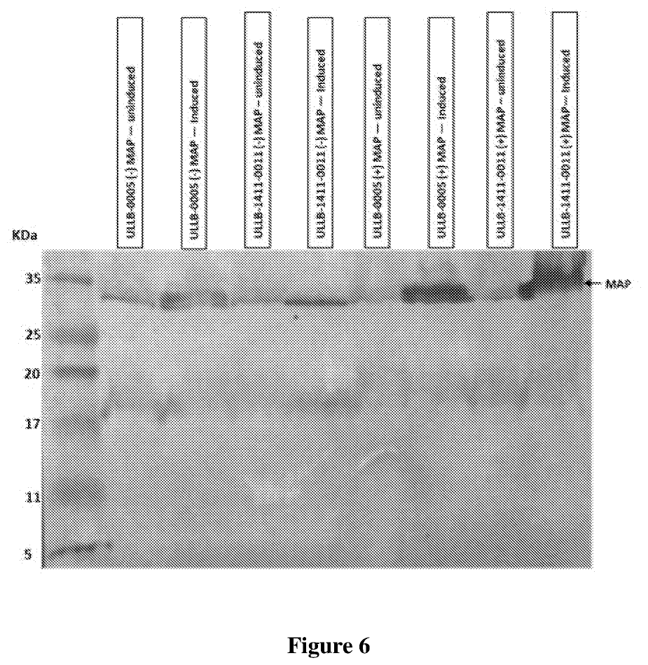

The results can be viewed in FIG. 6.

TABLE 1
Analysis of Expressed protein of SEQ ID NO: 10

| Sr# | Batch Number | Protein Yield | % Purity | % Met Content |
|---|---|---|---|---|
| 1 | Control | 0.87 | 86.29 | 13.18 |
| 2 | Batch 1 | 1.12 | 99.78 | 0.18 |
| 3 | Batch 2 | 0.93 | 99.72 | 0.28 |
| 4 | Batch 3 | 1.17 | 99.75 | 0.25 |

Conclusion: It was observed that expressed protein of SEQ ID NO: 10 obtained had not more than 2% of N terminal methionine. Similar observations were found for expressed protein of SEQ ID NO: 13 and SEQ ID NO: 16. The Western blot analysis revealed that upon induction of the expression vector, the band thickness was high for induced plasmids having expressed proteins of SEQ ID NO: 13 and SEQ ID NO: 16 along with Methionine aminopeptidase gene.

### Example 8: Analysis of Different Promoters for MAP Expression

The N terminal initiator methionine content of the expressed protein, wherein MAP gene was operably linked to one of the promoters selected from araE promoter, UV5 promoter and TAC promoter, were evaluated.

Purified protein of SEQ ID NO:10 (Protein Variant 1) obtained using three different promoters with MAP gene were analyzed for yield of expressed protein and the purity contents.

Expressed protein of SEQ ID NO: 10 (Protein Variant 1) in E coli without exogenous MAP gene expression system was used as control for analysis purpose.

| SI | Promoter sequence for | % Soluble | | % Methionine |
|---|---|---|---|---|
| NO: | MAP expression | expression | % Purity | Content |
| 1 | Control | ~60% | ~86 | 13.0% |
| 2 | araE promoter | ~65% | ~91 | 8.5% |
| 3 | UV5 promoter | ~60% | ~90 | 6.7% |
| 4 | TAC promoter | ~65% | ~96 | 1.25% |

Conclusion: Upon evaluation, it was observed that MAP gene expression under control of TAC promoter, yielded protein with not more than 2% of N terminal methionine content.

1. A recombinant plasmid comprising of:
a nucleotide sequence encoding Methionine Aminopeptidase (MAP) protein or a variant thereof, operably linked to a promoter sequence of SEQ ID NO 5; and
a nucleotide sequence encoding protein of interest operably linked to a promoter sequence of SEQ ID NO 7;
wherein the recombinant plasmid effects the protein of interest consisting of less than 2% N-terminal initiator methionine.

2. The recombinant plasmid as claimed in claim 1, wherein the recombinant plasmid is a modified pET27b plasmid.

3. The recombinant plasmid as claimed in claim 1, wherein the MAP protein or a variant thereof is further operably linked to a terminator.

4. The recombinant plasmid as claimed in claim 1, wherein the protein of interest is further operably linked to a terminator.

5. The recombinant plasmid as claimed in claim 1, wherein the nucleotide sequence encoding MAP protein operably linked to promoter sequence represented as SEQ ID NO: 2.

6. The recombinant plasmid as claimed in claim 1, wherein the protein of interest is a protein of SEQ ID NO: 10.

7. The recombinant plasmid as claimed in claim 1, wherein the protein of interest is a protein of SEQ ID NO: 13 or SEQ ID NO: 16.

8. The recombinant plasmid as claimed in claim 1, wherein the MAP protein is a protein of SEQ ID NO: 4 or a variant thereof.

9. The use of recombinant plasmid as claimed in claim 4, wherein the protein of interest can be used in cancer diagnosis or cancer therapy.

10. The use of recombinant plasmid as claimed in claim 1, wherein the recombinant plasmid can be used to produce protein of interest with less than 2% N terminal initiator methionine.

11. The recombinant plasmid as claimed in claim 1, wherein the nucleotide sequence encoding protein of interest operably linked to a promoter is represented as FIG. 4.

12. A recombinant plasmid of SEQ ID NO: 18, wherein the plasmid further comprises of a nucleotide sequence encoding protein of interest operably linked to a promoter sequence of SEQ ID NO: 7.

13. A host cell comprising recombinant plasmid as claimed in claim 1, wherein the host cell is a prokaryote.

14. The host cell as claimed in claim 13, wherein prokaryote is Escherichia coli DE3 strains.

15. A method for producing protein of interest, comprising steps of
a) providing a host cell with a recombinant plasmid as claimed in claim 1;
b) culturing the host cell in a culture medium;
c) purifying the protein of interest from the cultured host cell or the culture medium; and
wherein the purified protein consists of less than 2% N terminal initiator methionine.

16. The recombinant plasmid as claimed in claim 1, comprising
a nucleotide sequence of SEQ ID NO: 3 operably linked to a promoter sequence of SEQ ID NO: 5 and a terminator sequence; and
a nucleotide sequence encoding protein of interest operably linked to a promoter sequence of SEQ ID NO 7 and a terminator sequence;
wherein the recombinant plasmid effects the protein of interest consisting of less than 2% N terminal initiator methionine.


SEQUENCE LISTING (21 sequences)

SEQ ID NO: 1 - DNA, 5414 bp, synthetic construct
  source 1..5414: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgacatcctcggggtcttccggggcgagttctggctggctagcccgtttgatctcgagtgcggccgcaagcttgtcgacggagctcgaattcggatccgaattaattccgatatccatggccatcgccggctgggcagcgaggagcagcagaccagcagcagcggtcggcagcaggtatttcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 2 - DNA, 952 bp, synthetic construct
  source 1..952: mol_type = other DNA
ttgacaattaatcatcggctcgtataatgtgtggaattgtgagcggataacaatttcacacaggaaacagccagtccgtttaggtgttttcacgagcacttcaccaacaaggaccatagactagtatggctatctcaatcaagaccccagaagatatcgaaaaaatgcgcgtcgctggccgactggctgccgaagtgctggagatgatcgaaccgtatgttaaaccgggcgtcagcaccggcgagctggatcgcatctgtaatgattacattgttaatgaacaacacgcggtttctgcctgcctcggctatcacggctatccgaaatccgtttgcatctctattaatgaagtggtgtgccacggtatcccggacgatgctaagctgctgaaagatggcgatatcgttaacattgatgtcaccgtaatcaaagatggtttccacggcgatacctcgaaaatgtttatcgtcggtaagccgaccatcatgggcgaacgtctgtgccgcatcacgcaagaaagcctgtacctggcgctacgcatggtaaaaccaggcattaatctgcgcgaaatcggtgcggcgattcagaaatttgtcgaagcagaaggcttctccgtcgttcgtgaatattgcggacacggtattggtcgcggcttccatgaagaaccgcaggtgctgcactatgactcccgtgaaaccaacgtcgtactgaaacctgggatgacgttcaccatcgagccaatggtcaacgcgggtaaaaaagagatccgcaccatgaaagatggctggacggtaaaaaccaaagatcgcagcttgtctgcacaatatgagcatactattgtggtgactgataacggctgcgaaattctgacgctacgcaaggatgacaccatcccggcgataatctcgcacgacgaataataggctcaccttcacgggtgggcctttcttcg

SEQ ID NO: 3 - DNA, 797 bp, synthetic construct
  source 1..797: mol_type = other DNA
atggctatctcaatcaagaccccagaagatatcgaaaaaatgcgcgtcgctggccgactggctgccgaagtgctggagatgatcgaaccgtatgttaaaccgggcgtcagcaccggcgagctggatcgcatctgtaatgattacattgttaatgaacaacacgcggtttctgcctgcctcggctatcacggctatccgaaatccgtttgcatctctattaatgaagtggtgtgccacggtatcccggacgatgctaagctgctgaaagatggcgatatcgttaacattgatgtcaccgtaatcaaagatggtttccacggcgatacctcgaaaatgtttatcgtcggtaagccgaccatcatgggcgaacgtctgtgccgcatcacgcaagaaagcctgtacctggcgctacgcatggtaaaaccaggcattaatctgcgcgaaatcggtgcggcgattcagaaatttgtcgaagcagaaggcttctccgtcgttcgtgaatattgcggacacggtattggtcgcggcttccatgaagaaccgcaggtgctgcactatgactcccgtgaaaccaacgtcgtactgaaacctgggatgacgttcaccatcgagccaatggtcaacgcgggtaaaaaagagatccgcaccatgaaagatggctggacggtaaaaaccaaagatcgcagcttgtctgcacaatatgagcatactattgtggtgactgataacggctgcgaaattctgacgctacgcaaggatgacaccatcccggcgataatctcgcacgacgaataata

SEQ ID NO: 4 - protein, 264 aa, synthetic construct
MAISIKTPEDIEKMRVAGRLAAEVLEMIEPYVKPGVSTGELDRICNDYIVNEQHAVSACLGYHGYPKSVCISINEVVCHGIPDDAKLLKDGDIVNIDVTVIKDGFHGDTSKMFIVGKPTIMGERLCRITQESLYLALRMVKPGINLREIGAAIQKFVEAEGFSVVREYCGHGIGRGFHEEPQVLHYDSRETNVVLKPGMTFTIEPMVNAGKKEIRTMKDGWTVKTKDRSLSAQYEHTIVVTDNGCEILTLRKDDTIPAIISHDE

SEQ ID NO: 5 - DNA, 29 bp, synthetic construct
  source 1..29: mol_type = other DNA
ttgacaattaatcatcggctcgtataatg

SEQ ID NO: 6 - DNA, 30 bp, synthetic construct
  source 1..30: mol_type = other DNA
ggctcaccttcacgggtgggcctttcttcg

SEQ ID NO: 7 - DNA, 19 bp, synthetic construct
  source 1..19: mol_type = other DNA
taatacgactcactatagg

SEQ ID NO: 8 - DNA, 48 bp, synthetic construct
  source 1..48: mol_type = other DNA
ctagcataaccccttggggcctctaaacgggtcttgaggggttttttg

SEQ ID NO: 9 - DNA, 423 bp, synthetic construct
  source 1..423: mol_type = other DNA
acctataaaattaccgtgcgcgtgtatcagaccaacccggatgcctttttccatccggtggaaaaaaccgtgtggaaatatgcgaatggcggtacctggacgattacggatgatcagcatgtgctgacgatgggtggtagcggtaccagcggcaccctgcgttttcacgcagataatggcgaaagcttcaccgccacctttggtgtgcataattataaacgctggtgtgatattgtgaccaacctggcagcggatgaaaccggcatggttattaatcagcagtattatagtcagaaaaaccgcgaagaagcgcgtgaacgccagctgagtaactatcaggtgaaaaatgcgaaaggccgtaacttccagattgtttataccgaagcggaaggcaatgatctgcatgcgaacctgattatcggc

SEQ ID NO: 10 - protein, 141 aa, synthetic construct
TYKITVRVYQTNPDAFFHPVEKTVWKYANGGTWTITDDQHVLTMGGSGTSGTLRFHADNGESFTATFGVHNYKRWCDIVTNLAADETGMVINQQYYSQKNREEARERQLSNYQVKNAKGRNFQIVYTEAEGNDLHANLIIG

SEQ ID NO: 11 - DNA, 752 bp, synthetic construct
  source 1..752: mol_type = other DNA
taatacgactcactataggggaattgtgagcggataacaattcccctctagaaataattttgtttaactttaagaaggagatatacatatgacctataaaattaccgtgcgcgtgtatcagaccaacccggatgcctttttccatccggtggaaaaaaccgtgtggaaatatgcgaatggcggtacctggacgattacggatgatcagcatgtgctgacgatgggtggtagcggtaccagcggcaccctgcgttttcacgcagataatggcgaaagcttcaccgccacctttggtgtgcataattataaacgctggtgtgatattgtgaccaacctggcagcggatgaaaccggcatggttattaatcagcagtattatagtcagaaaaaccgcgaagaagcgcgtgaacgccagctgagtaactatcaggtgaaaaatgcgaaaggccgtaacttccagattgtttataccgaagcggaaggcaatgatctgcatgcgaacctgattatcggctaatgaggatccgaattcgagctccgtcgacaagcttgcggccgcactcgagatcaaacgggctagccagccagaactcgccccggaagaccccgaggatgtcgagcaccaccaccaccaccactgagatccggctgctaacaaagcccgaaaggaagctgagttggctgctgccaccgctgagcaataactagcataaccccttggggcctctaaacgggtcttgaggggttttttg

SEQ ID NO: 12 - DNA, 426 bp, synthetic construct
  source 1..426: mol_type = other DNA
agctataaaattaccgtgcgcgtgtatcagaccaacccggatgcctttttccatccggtggaaaaaaccgtgtggaaatatgcgaatggcggtacctggacgattacggatgatcagcatgtgctgacgatgggtggtagcggtaccagcggcaccctgcgttttcacgcagataatggcgaaagcttcaccgccacctttggtgtgcataattataaacgctggggcgatattgtgaccaacctggcagcggatgaaaccggcatggttattaatcagcagtattatagtcagaaaaaccgcgaagaagcgcgtgaacgccagctgagtaactatcaggtgaaaaatgcgaaaggccgtaacttccagattgtttataccgaagcggaaggcaatgatctgcatgcgaacctgattatcggctgc

SEQ ID NO: 13 - protein, 142 aa, synthetic construct
SYKITVRVYQTNPDAFFHPVEKTVWKYANGGTWTITDDQHVLTMGGSGTSGTLRFHADNGESFTATFGVHNYKRWGDIVTNLAADETGMVINQQYYSQKNREEARERQLSNYQVKNAKGRNFQIVYTEAEGNDLHANLIIGC

SEQ ID NO: 14 - DNA, 755 bp, synthetic construct
  source 1..755: mol_type = other DNA
taatacgactcactataggggaattgtgagcggataacaattcccctctagaaataattttgtttaactttaagaaggagatatacatatgagctataaaattaccgtgcgcgtgtatcagaccaacccggatgcctttttccatccggtggaaaaaaccgtgtggaaatatgcgaatggcggtacctggacgattacggatgatcagcatgtgctgacgatgggtggtagcggtaccagcggcaccctgcgttttcacgcagataatggcgaaagcttcaccgccacctttggtgtgcataattataaacgctggggcgatattgtgaccaacctggcagcggatgaaaccggcatggttattaatcagcagtattatagtcagaaaaaccgcgaagaagcgcgtgaacgccagctgagtaactatcaggtgaaaaatgcgaaaggccgtaacttccagattgtttataccgaagcggaaggcaatgatctgcatgcgaacctgattatcggctgctaatgaggatccgaattcgagctccgtcgacaagcttgcggccgcactcgagatcaaacgggctagccagccagaactcgccccggaagaccccgaggatgtcgagcaccaccaccaccaccactgagatccggctgctaacaaagcccgaaaggaagctgagttggctgctgccaccgctgagcaataactagcataaccccttggggcctctaaacgggtcttgaggggttttttg

SEQ ID NO: 15 - DNA, 429 bp, synthetic construct
  source 1..429: mol_type = other DNA
agctataaaattaccgtgcgcgtgtatcagaccaacccggatgcctttttccatccggtggaaaaaaccgtgtggaaatatgcgaatggcggtacctggacgattacggatgatcagcatgtgctgacgatgggtggtagcggtaccagcggcaccctgcgttttcacgcagataatggcgaaagcttcaccgccacctttggtgtgcataattataaacgctggggcgatattgtgaccaacctggcagcggatgaaaccggcatggttattaatcagcagtattatagtcagaaaaaccgcgaagaagcgcgtgaacgccagctgagtaactatcaggtgaaaaatgcgaaaggccgtaacttccagattgtttataccgaagcggaaggcaatgatctgcatgcgaacctgattatcggcagctgc

SEQ ID NO: 16 - protein, 143 aa, synthetic construct
SYKITVRVYQTNPDAFFHPVEKTVWKYANGGTWTITDDQHVLTMGGSGTSGTLRFHADNGESFTATFGVHNYKRWGDIVTNLAADETGMVINQQYYSQKNREEARERQLSNYQVKNAKGRNFQIVYTEAEGNDLHANLIIGSC

SEQ ID NO: 17 - DNA, 758 bp, synthetic construct
  source 1..758: mol_type = other DNA
taatacgactcactataggggaattgtgagcggataacaattcccctctagaaataattttgtttaactttaagaaggagatatacatatgagctataaaattaccgtgcgcgtgtatcagaccaacccggatgcctttttccatccggtggaaaaaaccgtgtggaaatatgcgaatggcggtacctggacgattacggatgatcagcatgtgctgacgatgggtggtagcggtaccagcggcaccctgcgttttcacgcagataatggcgaaagcttcaccgccacctttggtgtgcataattataaacgctggggcgatattgtgaccaacctggcagcggatgaaaccggcatggttattaatcagcagtattatagtcagaaaaaccgcgaagaagcgcgtgaacgccagctgagtaactatcaggtgaaaaatgcgaaaggccgtaacttccagattgtttataccgaagcggaaggcaatgatctgcatgcgaacctgattatcggcagctgctaatgaggatccgaattcgagctccgtcgacaagcttgcggccgcactcgagatcaaacgggctagccagccagaactcgccccggaagaccccgaggatgtcgagcaccaccaccaccaccactgagatccggctgctaacaaagcccgaaaggaagctgagttggctgctgccaccgctgagcaataactagcataaccccttggggcctctaaacgggtcttgaggggttttttg

SEQ ID NO: 18 - DNA, 6191 bp, synthetic construct
  source 1..6191: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgacatcctcggggtcttccggggcgagttctggctggctagcccgtttgatctcgagtgcggccgcaagcttgtcgacggagctcgaattcggatccgaattaattccgatatccatggccatcgccggctgggcagcgaggagcagcagaccagcagcagcggtcggcagcaggtatttcatatgtatatctccttcttaaagttaaacaaaattatttctagaggggaattgttatccgctcacaattcccctatagtgagtcgtattaatttcgcgggatcgagatctggtaccgagctgttgacaattaatcatcggctcgtataatgtgtggaattgtgagcggataacaatttcacacaggaaacagccagtccgtttaggtgttttcacgagcacttcaccaacaaggaccatagactagtatggctatctcaatcaagaccccagaagatatcgaaaaaatgcgcgtcgctggccgactggctgccgaagtgctggagatgatcgaaccgtatgttaaaccgggcgtcagcaccggcgagctggatcgcatctgtaatgattacattgttaatgaacaacacgcggtttctgcctgcctcggctatcacggctatccgaaatccgtttgcatctctattaatgaagtggtgtgccacggtatcccggacgatgctaagctgctgaaagatggcgatatcgttaacattgatgtcaccgtaatcaaagatggtttccacggcgatacctcgaaaatgtttatcgtcggtaagccgaccatcatgggcgaacgtctgtgccgcatcacgcaagaaagcctgtacctggcgctacgcatggtaaaaccaggcattaatctgcgcgaaatcggtgcggcgattcagaaatttgtcgaagcagaaggcttctccgtcgttcgtgaatattgcggacacggtattggtcgcggcttccatgaagaaccgcaggtgctgcactatgactcccgtgaaaccaacgtcgtactgaaacctgggatgacgttcaccatcgagccaatggtcaacgcgggtaaaaaagagatccgcaccatgaaagatggctggacggtaaaaaccaaagatcgcagcttgtctgcacaatatgagcatactattgtggtgactgataacggctgcgaaattctgacgctacgcaaggatgacaccatcccggcgataatctcgcacgacgaataataggctcaccttcacgggtgggcctttcttcggcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 19 - DNA, 6537 bp, synthetic construct
  source 1..6537: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgacatcctcggggtcttccggggcgagttctggctggctagcccgtttgatctcgagtgcggccgcaagcttgtcgacggagctcgaattcggatcctcattagccgataatcaggttcgcatgcagatcattgccttccgcttcggtataaacaatctggaagttacggcctttcgcatttttcacctgatagttactcagctggcgttcacgcgcttcttcgcggtttttctgactataatactgctgattaataaccatgccggtttcatccgctgccaggttggtcacaatatcacaccagcgtttataattatgcacaccaaaggtggcggtgaagctttcgccattatctgcgtgaaaacgcagggtgccgctggtaccgctaccacccatcgtcagcacatgctgatcatccgtaatcgtccaggtaccgccattcgcatatttccacacggttttttccaccggatggaaaaaggcatccgggttggtctgatacacgcgcacggtaattttataggtcatatgtatatctccttcttaaagttaaacaaaattatttctagaggggaattgttatccgctcacaattcccctatagtgagtcgtattaatttcgcgggatcgagatctggtaccgagctgttgacaattaatcatcggctcgtataatgtgtggaattgtgagcggataacaatttcacacaggaaacagccagtccgtttaggtgttttcacgagcacttcaccaacaaggaccatagactagtatggctatctcaatcaagaccccagaagatatcgaaaaaatgcgcgtcgctggccgactggctgccgaagtgctggagatgatcgaaccgtatgttaaaccgggcgtcagcaccggcgagctggatcgcatctgtaatgattacattgttaatgaacaacacgcggtttctgcctgcctcggctatcacggctatccgaaatccgtttgcatctctattaatgaagtggtgtgccacggtatcccggacgatgctaagctgctgaaagatggcgatatcgttaacattgatgtcaccgtaatcaaagatggtttccacggcgatacctcgaaaatgtttatcgtcggtaagccgaccatcatgggcgaacgtctgtgccgcatcacgcaagaaagcctgtacctggcgctacgcatggtaaaaccaggcattaatctgcgcgaaatcggtgcggcgattcagaaatttgtcgaagcagaaggcttctccgtcgttcgtgaatattgcggacacggtattggtcgcggcttccatgaagaaccgcaggtgctgcactatgactcccgtgaaaccaacgtcgtactgaaacctgggatgacgttcaccatcgagccaatggtcaacgcgggtaaaaaagagatccgcaccatgaaagatggctggacggtaaaaaccaaagatcgcagcttgtctgcacaatatgagcatactattgtggtgactgataacggctgcgaaattctgacgctacgcaaggatgacaccatcccggcgataatctcgcacgacgaataataggctcaccttcacgggtgggcctttcttcggcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 20 - DNA, 6540 bp, synthetic construct
  source 1..6540: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgacatcctcggggtcttccggggcgagttctggctggctagcccgtttgatctcgagtgcggccgcaagcttgtcgacggagctcgaattcggatcctcattagcagccgataatcaggttcgcatgcagatcattgccttccgcttcggtataaacaatctggaagttacggcctttcgcatttttcacctgatagttactcagctggcgttcacgcgcttcttcgcggtttttctgactataatactgctgattaataaccatgccggtttcatccgctgccaggttggtcacaatatcgccccagcgtttataattatgcacaccaaaggtggcggtgaagctttcgccattatctgcgtgaaaacgcagggtgccgctggtaccgctaccacccatcgtcagcacatgctgatcatccgtaatcgtccaggtaccgccattcgcatatttccacacggttttttccaccggatggaaaaaggcatccgggttggtctgatacacgcgcacggtaattttatagctcatatgtatatctccttcttaaagttaaacaaaattatttctagaggggaattgttatccgctcacaattcccctatagtgagtcgtattaatttcgcgggatcgagatctggtaccgagctgttgacaattaatcatcggctcgtataatgtgtggaattgtgagcggataacaatttcacacaggaaacagccagtccgtttaggtgttttcacgagcacttcaccaacaaggaccatagactagtatggctatctcaatcaagaccccagaagatatcgaaaaaatgcgcgtcgctggccgactggctgccgaagtgctggagatgatcgaaccgtatgttaaaccgggcgtcagcaccggcgagctggatcgcatctgtaatgattacattgttaatgaacaacacgcggtttctgcctgcctcggctatcacggctatccgaaatccgtttgcatctctattaatgaagtggtgtgccacggtatcccggacgatgctaagctgctgaaagatggcgatatcgttaacattgatgtcaccgtaatcaaagatggtttccacggcgatacctcgaaaatgtttatcgtcggtaagccgaccatcatgggcgaacgtctgtgccgcatcacgcaagaaagcctgtacctggcgctacgcatggtaaaaccaggcattaatctgcgcgaaatcggtgcggcgattcagaaatttgtcgaagcagaaggcttctccgtcgttcgtgaatattgcggacacggtattggtcgcggcttccatgaagaaccgcaggtgctgcactatgactcccgtgaaaccaacgtcgtactgaaacctgggatgacgttcaccatcgagccaatggtcaacgcgggtaaaaaagagatccgcaccatgaaagatggctggacggtaaaaaccaaagatcgcagcttgtctgcacaatatgagcatactattgtggtgactgataacggctgcgaaattctgacgctacgcaaggatgacaccatcccggcgataatctcgcacgacgaataataggctcaccttcacgggtgggcctttcttcggcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 21 - DNA, 6543 bp, synthetic construct
  source 1..6543: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgacatcctcggggtcttccggggcgagttctggctggctagcccgtttgatctcgagtgcggccgcaagcttgtcgacggagctcgaattcggatcctcattagcagctgccgataatcaggttcgcatgcagatcattgccttccgcttcggtataaacaatctggaagttacggcctttcgcatttttcacctgatagttactcagctggcgttcacgcgcttcttcgcggtttttctgactataatactgctgattaataaccatgccggtttcatccgctgccaggttggtcacaatatcgccccagcgtttataattatgcacaccaaaggtggcggtgaagctttcgccattatctgcgtgaaaacgcagggtgccgctggtaccgctaccacccatcgtcagcacatgctgatcatccgtaatcgtccaggtaccgccattcgcatatttccacacggttttttccaccggatggaaaaaggcatccgggttggtctgatacacgcgcacggtaattttatagctcatatgtatatctccttcttaaagttaaacaaaattatttctagaggggaattgttatccgctcacaattcccctatagtgagtcgtattaatttcgcgggatcgagatctggtaccgagctgttgacaattaatcatcggctcgtataatgtgtggaattgtgagcggataacaatttcacacaggaaacagccagtccgtttaggtgttttcacgagcacttcaccaacaaggaccatagactagtatggctatctcaatcaagaccccagaagatatcgaaaaaatgcgcgtcgctggccgactggctgccgaagtgctggagatgatcgaaccgtatgttaaaccgggcgtcagcaccggcgagctggatcgcatctgtaatgattacattgttaatgaacaacacgcggtttctgcctgcctcggctatcacggctatccgaaatccgtttgcatctctattaatgaagtggtgtgccacggtatcccggacgatgctaagctgctgaaagatggcgatatcgttaacattgatgtcaccgtaatcaaagatggtttccacggcgatacctcgaaaatgtttatcgtcggtaagccgaccatcatgggcgaacgtctgtgccgcatcacgcaagaaagcctgtacctggcgctacgcatggtaaaaccaggcattaatctgcgcgaaatcggtgcggcgattcagaaatttgtcgaagcagaaggcttctccgtcgttcgtgaatattgcggacacggtattggtcgcggcttccatgaagaaccgcaggtgctgcactatgactcccgtgaaaccaacgtcgtactgaaacctgggatgacgttcaccatcgagccaatggtcaacgcgggtaaaaaagagatccgcaccatgaaagatggctggacggtaaaaaccaaagatcgcagcttgtctgcacaatatgagcatactattgtggtgactgataacggctgcgaaattctgacgctacgcaaggatgacaccatcccggcgataatctcgcacgacgaataataggctcaccttcacgggtgggcctttcttcggcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca